(19) United States
(12) Patent Application Publication
(10) Pub. No.: US 2026/0338791 A1
(43) Pub. Date: Oct. 29, 2026

(54) SPR-BASED BINDING ASSAY FOR THE FUNCTIONAL ANALYSIS OF MULTIVALENT MOLECULES

(71) Applicant: Hoffmann-La Roche Inc., Branchburg, NJ (US)

(72) Inventors: Diana Angela PIPPIG, Muenchen (DE); Tilman SCHLOTHAUER, Penzberg (DE); Stefan SEEBER, Sindelsdorf (DE); Adrian ZWICK, Penzberg (DE)

(73) Assignee: Hoffmann-La Roche Inc., Branchburg, NJ (US)

(21) Appl. No.: 19/730,247

(22) Filed: Jul. 2, 2026

Related U.S. Application Data
(63) Continuation of application No. 17/488,649, filed on Sep. 29, 2021.
Application No. 17/488,649 is a continuation of application No. PCT/EP2020/058266, filed on Mar. 25, 2020.

(30) Foreign Application Priority Data
Mar. 29, 2019 (EP) 19166037.2

(51) Int. Cl. G01N 33/68; C07K 16/18; C07K 16/46; G01N 33/543

(52) U.S. Cl. CPC G01N 33/6854; C07K 16/18; C07K 16/468; G01N 33/54373; C07K 2317/31; C07K 2317/92

(57) ABSTRACT

Herein is reported a heterodimeric fusion polypeptide comprising a first proteinaceous moiety and a second proteinaceous moiety, wherein the first proteinaceous moiety and the second proteinaceous moiety are the first and the second antigen of a bispecific antibody which comprises a first binding site that specifically binds to the first proteinaceous moiety and a second binding site that specifically binds to the second proteinaceous moiety, wherein the first proteinaceous moiety is fused to the N-terminus of a first antibody heavy chain Fc-region polypeptide of the IgG1 subtype, wherein the second proteinaceous moiety is fused to the N-terminus of a second antibody heavy chain Fc-region polypeptide of the IgG1 subtype, wherein the first and the second heavy chain Fc-region polypeptide form a disulfide-linked heterodimer, wherein one or both of the heavy chain Fc-region polypeptides comprise a tag for immobilization to a solid phase at its C-terminus, and wherein the first and the second Fc-region polypeptide comprise the mutations T366W and T366S/L368A/Y407V, respectively, and the use of said fusion polypeptide for the determination of the avidity-based binding strength of a bispecific antibody, which comprises a first binding site specifically binding to a first antigen and a second binding site specifically binding to a second antigen, to said first and second antigen in a surface-plasmon-resonance-method.

### CROSS REFERENCE TO RELATED APPLICATIONS

This application is a continuation of U.S. application Ser. No. 17/488,649, filed Sep. 29, 2021, which is a continuation of International Application No. PCT/EP2020/058266, filed Mar. 25, 2020, which claims benefit to European Patent Application No. 19166037.2, filed Mar. 29, 2019, all of which are hereby expressly incorporated by reference in their entirety as though fully set forth herein.

The current invention is in the field of functional assays. Herein is reported a novel SPR-based binding assay for measuring the simultaneous interactions of multispecific antibodies with their different antigens. The method is especially suitable for the determination and measurement of the avidity-based binding strength of bispecific antibodies.

### BACKGROUND OF THE INVENTION

SPR (surface plasmon resonance) is a biosensor-based technology to measure real time protein-protein interaction. SPR technology has become a standard tool in biopharmaceutical research and development (see, e.g., M. A. Cooper, Nat. Rev. Drug Dis. 1 (2002) 515-528; D. G. Myszka, J. Mol. Recognit. 12 (1999) 390-408; R. L. Rich and D. G. Myszka, J. Mol. Recognit. 13 (2000) 388-407; D. G. Myszka and R. L. Rich, Pharm. Sci. Technol. Today 3 (2000) 310-317; R. Karlsson and A. Faelt, J. Immunol. Meth. 200 (1997) 121-133), and is commonly employed to determine rate constants for macromolecular interactions. The ability to determine association and dissociation kinetics for molecular interactions provides detailed insights into the mechanism of complex formation (see, e.g., T. A. Morton, D. G. Myszka, Meth. Enzymol. 295 (1998 ) 268-294). This information is becoming an essential part of the selection and optimization process for monoclonal antibodies and other biopharmaceutical products (see, e.g., K. Nagata and H. Handa, in Real-time analysis of biomolecular interactions, Springer, 2000; R. L. Rich and D. G. Myszka, Curr. Opin. Biotechnol. 11 (2000) 54-61; A. C. Malmborg and C. A. Borrebaeck, J. Immunol. Meth. 183 (1995 ) 7-13; W. Huber and F. Mueller, Curr. Pharm. Des. 12 (2006) 3999-4021). In addition, SPR technology allows the determination of the binding activity (binding capacity) of e.g. an antibody binding a target.

Only a couple of technologies are available which allow the functional assessment of two or more interactions in one approach (e.g. suspension array technology, reviewed in Y. Leng, Chem. Soc. Rev 44 (2015); time-resolved fluorescence assay (see, e.g., T-C. Liu, Clin. Biochem. 47 (2014 ) 439-444)). These technologies take advantage of the parallel detection of different fluorophores. In addition, optical biosensors exist, which allow the online- and therefore sequential-measurement of multiple interactions (see, e.g., D. G. Myszka, J. Mol. Recognit. 12 (1999) 390-408; R. L. Rich and D. G. Myszka, J. Mol. Recognit. 13 (2000) 388-407; D. G. Myszka and R. L. Rich, Pharm. Sci. Technol. Today 3 (2000) 310-317; R. Karlsson and A. Faelt, J. Immunol. Meth. 200 (1997) 121-133).

WO 2009/058564 disclosed a kinetic assay for measuring the binding kinetics of dimeric ligand (e.g., hCD80-mIg fusion protein or hCD86-mIg fusion protein) coated to sensor chips and dimeric analytes (e.g., mutant CTLA-4-Ig fusion proteins of the invention) in the mobile phase.

WO 2009/062942 disclosed a semi generic dual affinity polypeptide with different binding affinity toward the target and the capturing ligand respectively for use in chromatography. Binding domains which are specific and strong, but cannot be broken under normal elution conditions can be used in this invention.

WO 2010/112193 disclosed an SPR-based assay for the determination of simultaneous binding of a bispecific antibody <IGF-1R-EGFR> to EGFR and IGF1R wherein the bispecific antibody is immobilized to the chip.

WO 2011/143545 disclosed an SPR-based assay for the determination of simultaneous binding of two different antigens by a bispecific antibody wherein the bispecific antibody bridges between an immobilized antigen-Fc-fusion and a soluble antigen-Fc-fusion.

WO 2015/104406 disclosed the detection of binding affinities of multi-specific polypeptides to the respective targets, human Her2 and human CTLA-4, by Surface Plasmon Resonance wherein biotinylated multi-specific polypeptide was captured on the sensor chip.

Meschendoerfer, W. et al., disclosed SPR-based assays enable the full functional analysis of bispecific molecules (J. Pharm. Biomed. Anal. 132 (2016 ) 141-147).

WO 2016/082044 disclosed biparatopic anti-HER2 antibodies wherein the first antigen binding moiety and the second antigen binding moiety bind to different epitopes on the same antigen. For the determination of the binding of the individual paratopes of the biparatopic anti-HER2 antibody to monomeric and dimeric HER2 either HER2 ECD and an HER2-Fc fusion are immobilized on the sensor chip and contacted with the respective monovalent monospecific or bivalent monospecific antibody. In contrast thereto for the determination of cis- and trans-binding properties of the biparatopic bivalent antibody said antibody is immobilized on the sensor chip by an anti-human Fc antibody.

WO 2016/059068 disclosed VEGFR-2 binding polypeptides, especially dimeric maturated Z variants were produced and characterized using SPR analysis. The dimeric Z variants were also shown to be able to bind to VEGFR-2 expressed on the surface of mammalian cells.

WO 2017/027422 disclosed constructs having a SIRP-alpha domain or variant thereof. The SIRP-alpha polypeptides or constructs include a SIRP-alpha D1 variant fused to an Fc domain monomer, a human serum albumin (HSA), an albumin-binding peptide, or a polyethylene glycol (PEG) polymer.

US 2014/0193408 disclosed soluble proteins for use as therapeutics. It is especially disclosed as subject a soluble, multispecific, multivalent binding proteins comprising a complex of two heterodimers, wherein each heterodimer essentially consists of: (i) a first single chain polypeptide comprising: (a) an antibody heavy chain sequence having VH, CH1, CH2, and CH3 regions; and (b) a monovalent region of a mammalian binding molecule fused to the VH region; and (ii) a second single chain polypeptide comprising: (c) an antibody light chain sequence having a VL and CL region; and (d) a monovalent region of a mammalian binding molecule fused to the VL region; characterized in that each pair of VH and VL CDR sequences has specificity for an antigen, such that the total valency of said soluble protein is six.

US 2018/0009892 disclosed anti-ROR1 antibodies.

WO 2016/004383 disclosed tumor selective CTLA-4 antagonists.

### SUMMARY OF THE INVENTION

Herein is reported a novel binding assay for measuring the simultaneous interactions of multivalent antibodies with their different antigens. The method is especially suitable for the determination and measurement of the avidity-based binding strength of bispecific antibodies, including those bispecific antibodies binding to two different epitopes on the same antigen.

Herein is reported a novel binding assay for measuring the avidity-based binding strength, i.e. the gain in binding affinity based on avid binding, of an at least bivalent, bispecific antibody for binding with both of its binding sites simultaneously to the respective antigen or antigens. In one embodiment the binding assay is an ELISA or an SPR-based binding assay.

Herein is reported a novel binding assay for measuring the avidity-based binding strength, i.e. the gain in binding affinity based on avid binding, of an at least bivalent, bispecific antibody for binding to both of its targets/antigens simultaneously. In one embodiment the binding assay is an ELISA or an SPR-based binding assay.

The invention is based, at least in part, on the finding that avidity-based binding strength can be determined separated from affinity-based binding influence by using a method wherein the two antigens are immobilized (in case of an SPR-based method immobilization is on the chip surface and in case of an ELISA immobilization is on the solid phase) as heterodimeric molecule, i.e., e.g., with immobilized bispecific antigen, e.g. as bispecific Fc-fusion. In one preferred embodiment the method is a plasmon-resonance-based method and the two antigens are immobilized on the chip surface.

In setups according to the current invention with defined immobilization of the linked, different antigens, i.e. in setups which have an even distribution of the individual antigens, the bivalent, bispecific antibody will bind to both antigens simultaneously since the two antigens are properly spaced apart. This defined, simultaneous binding allows the determination of avid binding independent from affine binding. Furthermore, the dimeric/bispecific antigen displays a homogeneous and avid interaction at any immobilization level.

Immobilizing fused (differing) antigens, which are connected via linker, onto a biosensor chip creates an environment in which single copies of the two antigens are in very close proximity to each other, independent of the surface density of the fused heterodimeric antigen according to the current invention. This opens the way to new applications, such as, for example,
1) the kinetic assessment of avid interactions: The fused heterodimeric antigen according to the current invention allows the co-localization of the two targets of a bispecific antibody, even at very low surface densities of the fused heterodimeric antigen. Such a low surface density with defined proximity of the different antigens is required in order to determine kinetic binding parameters without limiting mass transfer or interfering rebinding (due to close proximity of a second copy of one of the antigens)
2) quality assessment of samples relative to a reference standard: Antibodies, capable of avid binding will always bind to both specificities in this setting, as the local concentration of the second binding partner is massively increased after the initial first binding event, due to the close proximity. Since the avidity effect has a huge impact on the dissociation rate constant kd of the antibody to its antigens, it is possible to assess the relative activity which correlates with the antibodies potency, simply by reading out a single response value in the dissociation phase of an SPR measurement. As the kd influences the equilibrium concentration, ELISA or similar methods can also utilize this approach to assess the bispecific binding capability of an antibody to all of its targets.

Differences between 1) and 2) are
the sample does not need to be/is not titrated in the case of 2;
only a single sample concentration is required to assess relative activity in case of 2;
the sample assessment does not rely on doing a kinetic fit.

One aspect of the invention is a method for determining the avidity-based binding strength of an at least bispecific binder, which comprises a first binding site specifically binding to a first antigen and a second binding site specifically binding to a second antigen, comprising the step of
determining the avidity-based binding strength of the bispecific binder from the change of the surface-plasmon-resonance-signal obtained by applying a solution comprising the bispecific binder to a solid phase to which a first-antigen-second-antigen-fusion-polypeptide is conjugated.

One aspect of the invention is a method for determining the avidity-based binding strength of an at least bispecific binder, which comprises a first binding site specifically binding to a first antigen and a second binding site specifically binding to a second antigen, to the first and second antigen, comprising the steps of
a) capturing a first-antigen-second-antigen-fusion-polypeptide on a solid phase and determining/measuring/establishing a first surface-plasmon-resonance-response,
b) applying to the solid phase of step a) a solution comprising the bispecific binder to form a captured first-antigen-second-antigen-fusion-polypeptide-bispecific-binder-complex and determining/measuring/establishing a second surface-plasmon-resonance-response,
c) determining/calculating from the difference between the first and the second surface-plasmon-resonance-response the avidity-based binding strength of the bispecific binder to the first and the second antigen.

One aspect of the invention is a method for determining the avidity-based binding strength of an at least bispecific binder, which comprises a first binding site specifically binding to a first antigen and a second binding site specifically binding to a second antigen, to the first and second antigen, comprising the steps of
a) capturing a first-antigen-second-antigen-fusion-polypeptide on a solid phase (and determining/measuring/establishing a baseline surface-plasmon-resonance-response),
b) applying to the solid phase of step a) a first solution comprising the bispecific binder at a first concentration to form a captured first-antigen-second-antigen-fusion-polypeptide-bispecific-binder-complex and determining/measuring/establishing a first surface-plasmon-resonance-response, (whereby the first surface-plasmon-resonance-response is the change of the surface-plasmon-resonance-response obtained by applying the bispecific binder to the solid phase to the baseline surface-plasmon-resonance-response,)
c) dissociating the captured first-antigen-second-antigen-fusion-polypeptide-bispecific-binder-complex and thereby regenerating the solid phase,
d) repeating steps b) and c) at least with a second solution comprising the bispecific binder at a second concentration and determining/measuring/establishing a second surface-plasmon-resonance-response, whereby all concentrations are different,
e) determining/calculating from the surface-plasmon-resonance-responses as determined in the previous steps the avidity-based binding strength of the bispecific binder to the first and the second antigen.

In one embodiment of all aspects and embodiments of the invention the at least bispecific binder is a bispecific antibody.

In one embodiment of all aspects and embodiments of the invention the at least bispecific antibody is a bispecific, bivalent antibody.

In one embodiment of all aspects and embodiments of the invention the at least bispecific antibody is a bispecific, trivalent antibody.

In one embodiment of all aspects and embodiments of the invention the at least bispecific antibody is a bispecific, tetravalent antibody.

In one embodiment of all aspects and embodiments of the invention the first antigen is at least a fragment of the first antigen comprising the epitope of the first binding site of the bispecific binder and the second antigen is at least a fragment of the second antigen comprising the epitope of the second binding site of the bispecific binder.

In one embodiment of all aspects and embodiments of the invention the first antigen and the second antigen are different.

In one embodiment of all aspects and embodiments of the invention the first and the second antigen are non-antibody antigens. The term “non-antibody antigen” denotes polypeptides that are not derived from an antibody, i.e. that are non-antibody proteins, i.e. that do not comprise any part of an antibody or a fragment thereof.

In one embodiment of all aspects and embodiments of the invention the solid phase is a surface plasmon resonance chip.

In one embodiment of all aspects and embodiments of the invention the second concentration differs from the first concentration by a factor of at least 2, 3, 4, 5 or 10.

One aspect of the invention is fusion-polypeptide of a first antigen and a second antigen of a bispecific binder.

Such a fusion protein according to the current invention is termed “first-antigen-second-antigen-fusion-polypeptide” herein.

In one embodiment of all aspects and embodiments of the invention the fusion-polypeptide according to the invention comprises as first antigen at least a fragment of the first antigen comprising the epitope of the first binding site of an at least bispecific binder and as second antigen at least a fragment of the second antigen comprising the epitope of the second binding site of an at least bispecific binder.

In one embodiment of all aspects and embodiments of the invention the fusion-polypeptide according to the invention is a linear polypeptide wherein the first antigen is at the C-terminus and the second antigen is at the N-terminus, or vice versa. In one embodiment the first antigen and the second antigen are connected by a peptidic linker. In one embodiment the peptidic linker comprises a tag for immobilization to a solid phase.

In one embodiment of all aspects and embodiments of the invention the fusion-polypeptide is a heterodimeric polypeptide comprising a first polypeptide, which is a fusion polypeptide of the first antigen and a first antibody heavy chain Fc-region polypeptide comprising a first set of heterodimerizing mutations, and a second polypeptide, which is a fusion polypeptide of the second antigen and a second antibody heavy chain Fc-region polypeptide comprising a second set of heterodimerizing mutations complementary to the first set of heterodimerizing mutations. In one embodiment the first antigen and the second antigen are at the N-terminus of the respective first or second Fc-region polypeptide. In one embodiment one or both of the Fc-region polypeptides comprise a tag for immobilization to a solid phase. In one embodiment the tag is at the C-terminus of the respective Fc-region polypeptide. In one embodiment the Fc-region is of the human IgG1 isotype. In one embodiment the first and the second set of heterodimerizing mutations are T366W and T366S/L368A/Y407V, respectively, or vice versa. In one embodiment the tag for immobilization is a histidine tag or a biotin.

In one embodiment of all aspects and embodiments of the invention the fusion-polypeptide is a heterodimeric polypeptide comprising a first polypeptide, which is a fusion polypeptide of the first antigen and a first antibody heavy chain constant region polypeptide comprising a first set of heterodimerizing mutations, and a second polypeptide, which is a fusion polypeptide of the second antigen and a second antibody heavy chain constant region polypeptide comprising a second set of heterodimerizing mutations complementary to the first set of heterodimerizing mutations. In one embodiment the first antigen and the second antigen are at the N-terminus of the respective first or second constant region polypeptide. In one embodiment one or both of the constant region polypeptides comprise a tag for immobilization to a solid phase. In one embodiment the tag is at the C-terminus of the respective Fc-region polypeptide. In one embodiment the Fc-region is of the human IgG1 isotype. In one embodiment the first and the second set of heterodimerizing mutations are T366W and T366S/L368A/Y407V, respectively, or vice versa. In one embodiment the tag for immobilization is a histidine tag or a biotin.

Thus, in general terms, one aspect of the current invention is a heterodimeric fusion polypeptide comprising
i) a first proteinaceous moiety, and
ii) a second proteinaceous moiety,
wherein
   the first proteinaceous moiety and the second proteinaceous moiety are
i) the first and the second antigen of an at least bispecific antibody which comprises a first binding site that specifically binds to the first proteinaceous moiety and a second binding site that specifically binds to the second proteinaceous moiety, or
ii) two copies of the same antigen of a bivalent, monospecific antibody,
   the first proteinaceous moiety is fused to the N-terminus of a first antibody heavy chain Fc-region polypeptide of the IgG1 subtype,
   the second proteinaceous moiety is fused to the N-terminus of a second antibody heavy chain Fc-region polypeptide of the IgG1 subtype,
   the first and the second heavy chain Fc-region polypeptide form a disulfide-linked heterodimer,
   one or both of the heavy chain Fc-region polypeptides comprise a tag for immobilization to a solid phase at its C-terminus, and
   the first and the second Fc-region polypeptide comprise the mutations T366W and T366S/L368A/Y407V, respectively.

In one embodiment of all aspects and embodiments of the invention the proteinaceous moiety is a polypeptide.

Such a heterodimeric fusion polypeptide is a fusion-polypeptide of a first antigen and a second antigen of an at least bispecific binder/antibody.

Likewise, such a heterodimeric fusion polypeptide is a fusion polypeptide of two copies of the antigen of a bivalent, monospecific binder/antibody.

In one embodiment of all aspects and embodiments of the invention the fusion-polypeptide according to the invention comprises only one, i.e. exactly one, first antigen and only one, i.e. exactly one, second antigen.

In one embodiment of all aspects and embodiments of the invention the first antigen and the second antigen are not the same polypeptide.

In one embodiment of all aspects and embodiments of the invention the first proteinaceous moiety and the second proteinaceous moiety are not from the same polypeptide.

One aspect of the invention is the use of the fusion-polypeptide of a first antigen and a second antigen of an at least bispecific binder according to the current invention in a surface-plasmon-resonance-method for the determination of the avidity-based binding strength of an at least bispecific binder, which comprises a first binding site specifically binding to a first antigen and a second binding site specifically binding to a second antigen, to said first and second antigen.

One aspect of the invention is the use of the fusion-polypeptide of a first antigen and a second antigen of an at least bispecific binder according to the current invention in an ELISA-method for the determination of the avidity-based binding strength of an at least bispecific binder, which comprises a first binding site specifically binding to a first antigen and a second binding site specifically binding to a second antigen, to said first and second antigen.

One aspect of the invention is an affinity chromatography column comprising the fusion-polypeptide of a first antigen and a second antigen of an at least bispecific binder according to the current invention as chromatography ligand.

One aspect of the invention is a method for separating/purifying an at least bispecific binder, which comprises a first binding site specifically binding to a first antigen and a second binding site specifically binding to a second antigen, with avidity-based binding to the first and second antigen from at least bispecific binders binding to the same first and second antigen without avidity-based binding, comprising the following steps:
a) applying a solution comprising at least bispecific binder with and without avidity-based binding to the first and second antigen to an affinity chromatography column according to the current invention,
b) recovering the at least bispecific binder with avidity-based binding to the first and second antigen from the column and thereby separating/purifying an at least bispecific binder from binder binding to the same first and second antigen without avidity-based binding.

One aspect of the invention is a method for purifying an at least bispecific binder, which comprises a first binding site specifically binding to a first antigen and a second binding site specifically binding to a second antigen, with avidity-based binding to the first and second antigen (from product- and/or process-related impurities), comprising the following steps:
a) applying a solution comprising the at least bispecific binder (and process- and/or product-related impurities) with avidity-based binding to the first and second antigen to an affinity chromatography column according to the current invention,
b) optionally washing the column whereby the at least bispecific binder with avidity-based binding to the first and second antigen remains bound to the column, and
c) recovering the at least bispecific binder with avidity-based binding to the first and second antigen from the column and thereby purifying an at least bispecific binder (from product- and/or process-related impurities).

One aspect of the current invention is a surface-plasmon-resonance-chip comprising the fusion-polypeptide of a first antigen and a second antigen of an at least bispecific binder according to the current invention immobilized in at least one flow cell.

One aspect according to the current invention is a method for producing a surface-plasmon-resonance-chip comprising the fusion-polypeptide of a first antigen and a second antigen of an at least bispecific binder according to the current invention immobilized in at least one flow cell, comprising the following step
immobilizing either directly or via a specific binding pair the fusion-polypeptide of a first antigen and a second antigen of an at least bispecific binder according to the current invention in at least one flow cell of the surface-plasmon-resonance-chip.

One aspect according to the current invention is a method for assessing the quality of a sample comprising an at least bivalent, bispecific antibody comprising the following steps:
separately applying solutions comprising a covalent fusion polypeptide, which comprises at one terminus the first antigen and at a different, second terminus the second antigen of the bivalent, bispecific antibody, at different concentrations to an SPR chip on which the bivalent, bispecific antibody has been immobilized and monitoring the SPR-signal thereafter, or vice versa,
and
comparing the determined readout with a reference sample and thereby determining the quality of the sample comprising the at least bivalent, bispecific antibody,
wherein the at least bivalent, bispecific antibody comprises a first binding site specifically binding to a first, non-antibody antigen and a second binding site specifically binding to a second, different, non-antibody antigen.

In one embodiment the method comprises the following steps
separately applying solutions comprising a covalent fusion polypeptide, which comprises at one terminus the first antigen and at a different, second terminus the second antigen of the bivalent, bispecific antibody, at different concentrations to an SPR chip on which the bivalent, bispecific antibody has been immobilized and monitoring the SPR-signal thereafter, or vice versa,
plotting the binding response (in resonance units) against the respective sample concentration,
fitting the data points of the obtained plot using a 2-parametric line fit and determining the y-axis intercept as readout,
comparing the determined readout by parallel-line transformation with that of a reference sample that has been analyzed and processed in the same way,
thereby determining the quality/purity/homogeneity of the sample comprising the at least bivalent, bispecific antibody,
wherein the at least bivalent, bispecific antibody comprises a first binding site specifically binding to a first, non-antibody antigen and a second binding site specifically binding to a second, different, non-antibody antigen.

One aspect according to the current invention is a method for selecting a cell line producing/expressing/secreting an at least bivalent, bispecific antibody comprising the following steps:
providing the individual supernatants of the separate cultivations of the cell lines of a multitude of recombinant mammalian cell lines producing/expressing/secreting an (heterologous) at least bivalent, bispecific antibody,
for each cell line separately applying solutions comprising a covalent fusion polypeptide, which comprises at one terminus the first antigen and at a different, second terminus the second antigen of the bivalent, bispecific antibody, at different concentrations to an SPR chip on which the bivalent, bispecific antibody from the cultivation supernatant of said cell line has been immobilized and monitoring the SPR-signal thereafter, or vice versa
comparing the determined readouts with each other and thereby determining the relative quality of the at least bivalent, bispecific antibody produced by each cell line,
and
selecting at least one cell line based on the relative quality of the at least bivalent, bispecific antibody produced,
wherein the at least bivalent, bispecific antibody comprises a first binding site specifically binding to a first, non-antibody antigen and a second binding site specifically binding to a second, different, non-antibody antigen.

In one embodiment the method comprises the following steps
providing the individual supernatants of the separate cultivations of the cell lines of a multitude of recombinant mammalian cell lines producing/expressing/secreting an (heterologous) at least bivalent, bispecific antibody,
for each cell line separately applying solutions comprising a covalent fusion polypeptide, which comprises at one terminus the first antigen and at a different, second terminus the second antigen of the bivalent, bispecific antibody, at different concentrations to an SPR chip on which the bivalent, bispecific antibody from the cultivation supernatant of said cell line has been immobilized and monitoring the SPR-signal thereafter, or vice versa,
plotting the binding response (in resonance units) against the respective sample concentration,
fitting the data points of the obtained plot using a 2-parametric line fit and determining the y-axis intercept as readout, thereby determining the relative quality of the at least bivalent, bispecific antibody produced by each cell line, and
selecting at least one cell line based on the relative quality of the at least bivalent, bispecific antibody produced,
wherein the at least bivalent, bispecific antibody comprises a first binding site specifically binding to a first, non-antibody antigen and a second binding site specifically binding to a second, different, non-antibody antigen.

All the methods as outlined before using SPR can be likewise adopted to an ELISA format wherein the readout is the assay signal (color intensity) instead of the SPR-signal.

### Description of Specific Embodiments of the Invention

Herein is reported a novel SPR-based binding assay for measuring the avidity-based binding strength, i.e. the gain in binding affinity based on avid binding, of an at least bivalent or at least bispecific antibody for simultaneous binding with both of its binding sites or to two of its targets/antigens, respectively.

The invention is based, at least in part, on the finding that avidity-based binding strength can be determined separated from affinity-based binding influence by using a surface-plasmon-resonance-based method, wherein the antigen of a bivalent, monospecific antibody is immobilized on the chip surface as dimer, i.e. with immobilized bivalent, dimeric antigen, e.g. as dimeric Fc-fusion.

The invention is based, at least in part, on the finding that avidity-based binding strength can be determined separated from affinity-based binding influence by using a surface-plasmon-resonance-based method, wherein the two antigens of an at least bispecific antibody are immobilized on the chip surface as bispecific molecule, i.e. with immobilized bispecific antigen, e.g. as bispecific Fc-fusion.

The invention is based, at least in part, on the finding that by presenting the antigen of a bivalent, monospecific antibody in covalently linked form on the surface of an SPR-chip it is possible to determine the avidity gained by the simultaneous binding with both binding sites.

The invention is based, at least in part, on the finding that by presenting the two antigens of an at least bispecific binder in covalently linked form on the surface of an SPR-chip it is possible to determine the avidity gained by the simultaneous bispecific binding.

The invention is further based, at least in part, on the finding that with the SPR assay according to the current invention an avidity driven binding improvement of bivalent, monospecific or at least bispecific binders can be determined. This concept allows for the selection of, e.g., bispecific binders with increased target specificity and thereby reduced off-target binding and side-effects.

### Definitions

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, MD (1991). The amino acid positions of all constant regions and domains of the heavy and light chain can be numbered according to the Kabat numbering system described in Kabat, et al., Sequences of Proteins of Immunological Interest, 5th ed., Public Health Service, National Institutes of Health, Bethesda, MD (1991) and is referred to as “numbering according to Kabat” herein. Specifically, the Kabat numbering system (see pages 647-660) of Kabat, et al., Sequences of Proteins of Immunological Interest, 5th ed., Public Health Service, National Institutes of Health, Bethesda, MD (1991) is used for the light chain constant domain CL of kappa and lambda isotype, and the Kabat EU index numbering system (see pages 661-723) is used for the constant heavy chain domains (CH1, Hinge, CH2 and CH3, which is herein further clarified by referring to “numbering according to Kabat EU index” in this case).

The term “about” denotes a range of +/−20% of the thereafter following numerical value. In one embodiment the term about denotes a range of +/−10% of the thereafter following numerical value. In one embodiment the term about denotes a range of +/−5% of the thereafter following numerical value.

“Affinity” or “binding affinity” refers to the strength of the sum total of non-covalent interactions between a single binding site of a molecule (e.g. an antibody) and its binding partner (e.g. an antigen). Unless indicated otherwise, as used herein, “binding affinity” refers to intrinsic binding affinity which reflects a 1:1 interaction between members of a binding pair (e.g. antibody and antigen). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by common methods known in the art, including those described herein. A particular method for measuring affinity is Surface Plasmon Resonance (SPR).

The term “antibody” herein is used in the broadest sense and encompasses various antibody structures, including but not limited to monoclonal antibodies, multispecific antibodies (e.g. bispecific antibodies, trispecific antibodies), and antibody fragments so long as they exhibit the desired antigen-binding activity.

An antibody in general comprises two so called light chain polypeptides (light chain) and two so called heavy chain polypeptides (heavy chain). Each of the heavy and light chain polypeptides contains a variable domain (variable region) (generally the amino terminal portion of the polypeptide chain) comprising binding regions that are able to interact with an antigen. Each of the heavy and light chain polypeptides comprises a constant region (generally the carboxyl terminal portion). The constant region of the heavy chain mediates the binding of the antibody i) to cells bearing a Fc gamma receptor (FcγR), such as phagocytic cells, or ii) to cells bearing the neonatal Fc receptor (FcRn) also known as Brambell receptor. It also mediates the binding to some factors including factors of the classical complement system such as component (C1q). The constant domains of an antibody heavy chain comprise the CH1-domain, the CH2-domain and the CH3-domain, whereas the light chain comprises only one constant domain, CL, which can be of the kappa isotype or the lambda isotype.

The variable domain of an immunoglobulin's light or heavy chain in turn comprises different segments, i.e. four framework regions (FR) and three hypervariable regions (HVR).

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “binding (to an antigen)” denotes the binding of an antibody to its antigen in an in vitro assay, in one embodiment in a binding assay in which the antibody is bound to a surface and binding of the antigen to the antibody is measured by Surface Plasmon Resonance (SPR). Binding means the measurement of the binding capacity of e.g. the antibody for target A or target B, or for a capture molecule e.g. anti-human-Fab-capture for the antibody.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1,L2, L3). Exemplary HVRs herein include:
(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, MD (1991));
(c) antigen contacts occurring at amino acid residues 27c-36 (L1), 46-55 (L2), 89-96(L3), 30-35b (H1), 47-58 (H2), and 93-101 (H3) (MacCallum et al. J. Mol. Biol. 262:732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al.

The term “valent” as used within the current application denotes the presence of a specified number of binding sites in a (antibody) molecule. As such, the terms “bivalent”, “tetravalent”, and “hexavalent” denote the presence of two binding site, four binding sites, and six binding sites, respectively, in a (antibody) molecule. The bispecific antibodies as reported herein are in one preferred embodiment “bivalent”.

The term “binding affinity” denotes the strength of the interaction of a single binding site with its respective target. Experimentally, the affinity can be determined e.g. by measuring the kinetic constants for association (kA) and dissociation (kD) of the antibody and the antigen in the equilibrium (see FIG. 2).

The term “binding avidity” denotes the combined strength of the interaction of multiple binding sites of one molecule (antibody) with the same target. As such, avidity is the combined synergistic strength of bond affinities rather than the sum of bonds. Requisites for avidity are: polyvalency of a molecule, such as an antibody, or of a functional multimer to one target (antigen), —multiple accessible epitopes on one soluble target OR multiple binding of an antibody to one epitope each on various immobilized targets.

The complex association does not differ between affine and avid binding. However, the complex dissociation for avid binding depends on the simultaneous dissociation of all binding sites involved. Therefore, the increase of binding strength due to avid binding (compared to affine binding) depends on the dissociation kinetics/complex stability: the bigger (higher) the complex stability, the less likely is the simultaneous dissociation of all involved binding sites; for very stable complexes, the difference of affine vs. avid binding becomes essentially zero; —the smaller (lower) the complex stability, the more likely is the simultaneous dissociation of all involved binding sites; the difference of affine vs. avid binding is increased.

### Multispecific Antibodies

In certain embodiments, the antibody is a multispecific antibody, e.g. a bispecific antibody. Multispecific antibodies are monoclonal antibodies that have binding specificities for at least two different sites. In certain embodiments, one of the binding specificities is for a first antigen and the other is for a different second antigen. In certain embodiments, multispecific antibodies may bind to two different epitopes of the same antigen. Multispecific antibodies may also be used to localize cytotoxic agents to cells, which express the antigen. Multispecific antibodies can be prepared as full-length antibodies or antibody fragments.

Techniques for making multispecific antibodies include, but are not limited to, recombinant co-expression of two immunoglobulin heavy chain-light chain pairs having different specificities (see Milstein, C. and Cuello, A. C., Nature 305 (1983) 537-540, WO 93/08829, and Traunecker, A., et al., EMBO J. 10 (1991) 3655-3659), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004); cross-linking two or more antibodies or fragments (see, e.g., U.S. Pat. No. 4,676,980, and Brennan, M., et al., Science 229 (1985) 81-83); using leucine zippers to produce bi-specific antibodies (see, e.g., Kostelny, S. A., et al., J. Immunol. 148(1992 ) 1547-1553; using “diabody” technology for making bispecific antibody fragments (see, e.g., Holliger, P., et al., Proc. Natl. Acad. Sci. USA 90 (1993) 6444-6448); and using single-chain Fv (scFv) dimers (see, e.g., Gruber, M., et al., J. Immunol. 152 (1994 ) 5368-5374); and preparing trispecific antibodies as described, e.g., in Tutt, A., et al., J. Immunol. 147 (1991) 60-69).

The antibody or fragment can also be a multispecific antibody as described in WO 2009/080251, WO 2009/080252, WO 2009/080253, WO 2009/080254, WO 2010/112193, WO 2010/115589, WO 2010/136172, WO 2010/145792, or WO 2010/145793.

The antibody or fragment thereof may also be a multispecific antibody as disclosed in WO 2012/163520 (also referred to as “DutaFab”).

Bispecific antibodies are generally antibody molecules that specifically bind to two different, non-overlapping epitopes on the same antigen or to two epitopes on different antigens.

Different bispecific antibody formats are known.

Exemplary bispecific antibody formats for which the methods as reported herein can be used are
the domain-exchanged format: a multispecific IgG antibody comprising a first Fab fragment and a second Fab fragment, wherein in the first Fab fragment
   a) only the CH1 and CL domains are replaced by each other (i.e. the light chain of the first Fab fragment comprises a VL and a CH1 domain and the heavy chain of the first Fab fragment comprises a VH and a CL domain);
   b) only the VH and VL domains are replaced by each other (i.e. the light chain of the first Fab fragment comprises a VH and a CL domain and the heavy chain of the first Fab fragment comprises a VL and a CH1 domain); or
   c) the CH1 and CL domains are replaced by each other and the VH and VL domains are replaced by each other (i.e. the light chain of the first Fab fragment comprises a VH and a CH1 domain and the heavy chain of the first Fab fragment comprises a VL and a CL domain); and
wherein the second Fab fragment comprises a light chain comprising a VL and a CL domain, and a heavy chain comprising a VH and a CH1 domain;
the domain exchanged antibody may comprise a first heavy chain including a CH3 domain and a second heavy chain including a CH3 domain, wherein both CH3 domains are engineered in a complementary manner by respective amino acid substitutions, in order to support heterodimerization of the first heavy chain and the modified second heavy chain, e.g. as disclosed in WO 96/27011, WO 98/050431, EP 1870459, WO 2007/110205, WO 2007/147901, WO 2009/089004, WO 2010/129304, WO 2011/90754, WO 2011/143545, WO 2012/058768, WO 2013/157954, or WO 2013/096291 (incorporated herein by reference);
the one-armed single chain format (=one-armed single chain antibody): antibody comprising a first binding site that specifically binds to a first epitope or antigen and a second binding site that specifically binds to a second epitope or antigen, whereby the individual chains are as follows
   light chain (variable light chain domain+light chain kappa constant domain)
   combined light/heavy chain (variable light chain domain+light chain constant domain+peptidic linker+variable heavy chain domain+CH1+Hinge+CH2+CH3 with knob mutation)
   heavy chain (variable heavy chain domain+CH1+Hinge+CH2+CH3 with hole mutation);
the two-armed single chain format (=two-armed single chain antibody): antibody comprising a first binding site that specifically binds to a first epitope or antigen and a second binding site that specifically binds to a second epitope or antigen, whereby the individual chains are as follows
   combined light/heavy chain 1 (variable light chain domain+light chain constant domain+peptidic linker+variable heavy chain domain+CH1+Hinge+CH2+CH3 with hole mutation)
   combined light/heavy chain 2 (variable light chain domain+light chain constant domain+peptidic linker+variable heavy chain domain+CH1+Hinge+CH2+CH3 with knob mutation);
the common light chain bispecific format (=common light chain bispecific antibody): antibody comprising a first binding site that specifically binds to a first epitope or antigen and a second binding site that specifically binds to a second epitope or antigen, whereby the individual chains are as follows
   light chain (variable light chain domain+light chain constant domain)
   heavy chain 1 (variable heavy chain domain+CH1+Hinge+CH2+CH3 with hole mutation)
   heavy chain 2 (variable heavy chain domain+CH1+Hinge+CH2+CH3 with knob mutation).

In one embodiment of all aspects and embodiments of the invention the bispecific antibody is a domain exchanged antibody.

In one embodiment of all aspects and embodiments of the invention the bispecific antibody is a one-armed single chain antibody.

In one embodiment of all aspects and embodiments of the invention the bispecific antibody is a two-armed single chain antibody.

In one embodiment of all aspects and embodiments of the invention the bispecific antibody is a common light chain bispecific antibody.

### State of the Art Surface-plasmon-resonance Methods

Kinetic binding parameters of antibodies to the respective antigens can be investigated by surface plasmon resonance, e.g. using a BIAcore instrument (GE Healthcare Biosciences AB, Uppsala, Sweden).

Briefly, for affinity measurements an anti-IgG antibody, e.g. an anti-human IgG or an anti-mouse IgG antibody, is immobilized on a CM5 chip via amine coupling for capture and presentation of the respective antibodies to be analyzed.

For example, about 2000 -12000 response units (RU) of a 10-30 μg/ml anti-IgG antibody is coupled onto some spots of the flow cells (e.g. spots 1 and 5 are active and spots 2and 4 are reference spots, or spots 1 and 2 are reactive and spots 3 and 4 are reference spots, etc.) of a CM5 sensor chip in a BIAcore B4000 instrument at pH 5.0 at 10-30 μl/min: by using an amine coupling kit supplied by GE Healthcare.

Binding is measured in HBS buffer (HBS-P (10 mM HEPES, 150 mM NaCl, 0.005% Tween 20, pH 7.4), or HBS-EP+ (0.01 M HEPES, 0.15 M NaCl, 3 mM EDTA, 0.05 % v/v Surfactant PS20, pH 7.4), or HBS-ET (10 mM HEPES pH 7.4, 150 mM NaCl, 3 mM EDTA, 0.005% w/v Tween 20)), 25 °C (or alternatively at a different temperature in the range from 12 °C to 37 °C).

Thereafter the antibody was injected for 30 seconds with a concentration in the range of 10 nM to 1 μM and bound to the reactive spots of each flow cell.

Then the corresponding antigens are added in various concentrations in solution, such as e.g. 144 nM, 48 nM, 16 nM, 5.33 nM, 1.78 nM, 0.59 nM, 0.20 nM and 0 nM, depending on the affinity of the antibody.

Association is measured by an antigen injection of 20 seconds to 10 minutes at 10-30 μl/min. flow rate.

Dissociation is measured by washing the chip surface with the respective buffer for 3-10 minutes.

A KD value is estimated using a 1:1 Langmuir binding model using the manufacturer's software and instructions. Negative control data (e.g. buffer curves) are subtracted from sample curves for correction of system intrinsic baseline drift and for noise signal reduction.

### The Method According to the Current Invention

The increasing complexity of novel biotherapeutics such as bispecific antibodies or fusion proteins raises new challenges for functional characterization. When compared to standard antibodies, two individual interactions need to be considered for bispecific monoclonal antibodies.

Herein is reported a novel SPR-based binding assay for measuring the avidity-based binding strength, i.e. the gain in binding affinity based on avid binding, of an at least bispecific antibody for binding to two of its targets/antigens simultaneously.

The invention is based, at least in part, on the finding that avidity-based binding strength can be determined separated from affinity-based binding influence by using a surface-plasmon-resonance-based method wherein two antigens are immobilized on the chip surface as bispecific molecule, i.e. with an immobilized bispecific antigen, e.g. as bispecific Fc-fusion.

Prior to the current invention the determination/measurement of avidity-based activity of bispecific binders, such as bispecific antibodies, was not possible. The determination of the binding of the isolated monospecific binding sites provides only isolated binding affinity values but did not allow access to binding avidity values. This is also the case when a mixture of the antigens is applied to a sensor surface to which the bispecific binder, e.g. bispecific antibody, has been immobilized.

Furthermore, prior to the current invention the determination/measurement of isolated avidity-based binding strength, i.e. defined avidity-binding-values, was not possible. For example, the use of sensor surfaces with random distribution of the antigens did not allow the determination/measurement of defined avidity-based binding strength as the obtained results were compromised by the influence of affine binding. Although the heterogeneity of the interaction decreases with increasing capture reagent density on the chip surface, i.e. immobilization level, a homogeneous interaction on a random distribution chip can only be achieved at high densities (response levels), i.e. antigen immobilization levels, at which kinetic evaluation of the binding interactions is not possible.

Thus, in SPR setups with random immobilization of the individual antigens, i.e. which have an uneven distribution of the different antigens, there will be antibodies that bind only to one antigen since the two antigens are too far apart. This non-defined, non-simultaneous binding results in the determination of a mixture of affine and avid binding (see FIG. 2A).

In contrast thereto, in SPR setups according to the current invention with defined immobilization of linked antigens, i.e. which have an even distribution of the different antigens, the antibodies will bind to both antigens simultaneously since the two antigens are properly spaced apart. This defined, simultaneous binding allows the determination of avid binding (FIG. 2B). Furthermore, the bispecific antigen displays a homogeneous and avid interaction at any immobilization level.

The invention is based, at least in part, on the finding that by presenting two antigens of an at least bispecific binder in covalently linked form on the surface of an SPR-chip it is possible to determine the avidity gained by simultaneous bispecific binding.

The invention is further based, at least in part, on the finding that with the SPR assay according to the current invention an avidity driven selectivity gain (ADSG) of at least bispecific binders can be determined. This concept allows for the selection of at least bispecific binders with increased target specificity and thereby reduced off-target binding and side-effects.

It has been found that by determining and comparing the dissociation constants of the at least bispecific binder (sample) and their individual targets with the dissociation constant for the bispecific target the avidity gain provided by binding with all valences at the same time can be assessed.

One aspect of the invention is a method for determining the avidity-based binding strength of an at least bispecific binder, which comprises a first binding site specifically binding to a first antigen and a second binding site specifically binding to a second antigen, comprising the step of
determining the avidity-based binding strength of the at least bispecific binder from the change of the surface-plasmon-resonance-signal obtained by applying a solution comprising the at least bispecific binder to a solid phase to which a first-antigen-second-antigen-fusion-polypeptide is conjugated.

When applying the standard approaches known from the art the avidity of the simultaneous binding of an at least bispecific antibody to its two antigens cannot be determined as the obtained results are not conclusive.

FIG. 3 shows schematically the surface of an SPR chip generally used for the determination of avid binding of an at least bispecific antibody. Two antigens are immobilized unlinked. For capture each antigen comprises a tag and is captured using an anti-tag antibody. Thereby a random surface is generated comprising a mixture of anti-tag antibody either only with the first antigen or only with the second antigen or with both antigens. Thus, no homogeneous surface but an uneven distribution of the antigens on the surface is obtained.

The result of this uneven distribution of the antigens over the SPR chip surface is that no homogeneous binding can be observed. FIG. 4 shows the different binding modes that result from the uneven distribution of the antigens on the chip surface: 1 and 2: binding with one binding site only; 3: simultaneous binding with two binding sites to a single complex; 4: simultaneous binding with two binding sites to two different complexes. Thus, at best it can be expected that at most 50% of the bispecific antibodies bind simultaneously and at most 25% of the antibodies bind to a single bispecific antigen complex.

Thus, the uneven distribution of the antigens on the surface lead to a mixture of binding events of the at least bispecific antibody which in turn results in non-conclusive sensograms. FIG. 5 shows the overlay of two sensograms obtained under identical conditions with the same bispecific antibody differing only in the format of the antigens captured on the chip surface: one was obtained with a mixture of the antigens according to a reference state of the art method, whereas the other was obtained with a fusion-polypeptide of the first and the second antigen with a method according to the current invention. It can be seen that the sensograms are strikingly different. Only in the sensogram obtained with a method according to the current invention using a fusion-polypeptide of the first and second antigen a homogeneous and analyzable response curve is obtained (FIG. 6). In the sensogram obtained with the reference method a different, overlaying dissociation processes (and thereby also kinetics) can be seen, which results in a non-analyzable response curve.

From FIG. 7 (same experimental conditions as for FIG. 5; difference is only the immobilized format of the antigen) it can be seen that the non-homogeneous dissociation kinetics obtained with the mixture of antigens is a result of the mixture of avid and affine binding events as the same sensogram is obtained by using only one of the antigens.

The sensograms in FIGS. 5 and 7 have been obtained with low capture levels of 5 -15 RU. Increasing the capture level, e.g. to 50-120 RU (FIG. 8) or even to 300-600 RU (FIG. 9), does not change the sensogram.

The same result can be seen when analyzing the interaction using kinetic rate maps. From FIG. 10 it can be seen that only in case a method according to the current invention is applied the uncompromised avid binding event can be analyzed.

One aspect of the invention is a method for determining the avidity-based binding strength of an at least bispecific binder, which comprises a first binding site specifically binding to a first antigen and a second binding site specifically binding to a second antigen, to the first and second antigen, comprising the steps of
a) capturing a first-antigen-second-antigen-fusion-polypeptide on a solid phase and determining/measuring/establishing a first surface-plasmon-resonance-response,
b) applying to the solid phase of step a) a solution comprising the at least bispecific binder to form a captured first-antigen-second-antigen-fusion-polypeptide-bispecific-binder-complex and determining/measuring/establishing a second surface-plasmon-resonance-response,
c) determining/calculating from the difference between the first and the second surface-plasmon-resonance-response the avidity-based binding strength of the at least bispecific binder to the first and the second antigen.

One aspect of the invention is a method for determining the avidity-based binding strength of an at least bispecific binder, which comprises a first binding site specifically binding to a first antigen and a second binding site specifically binding to a second antigen, to the first and second antigen, comprising the steps of
a) capturing a first-antigen-second-antigen-fusion-polypeptide on a solid phase (and determining/measuring/establishing a baseline surface-plasmon-resonance-response),
b) applying to the solid phase of step a) a first solution comprising the at least bispecific binder at a first concentration to form a captured first-antigen-second-antigen-fusion-polypeptide-bispecific-binder-complex and determining/measuring/establishing a first surface-plasmon-resonance-response, (whereby the first surface-plasmon-resonance-response is the change of the surface-plasmon-resonance-response obtained by applying the bispecific binder to the solid phase to the baseline surface-plasmon-resonance-response,)
c) dissociating the captured first-antigen-second-antigen-fusion-polypeptide-bispecific-binder-complex and thereby regenerating the solid phase,
d) repeating steps b) and c) at least with a second solution comprising the at least bispecific binder at a second concentration and determining/measuring/establishing a second surface-plasmon-resonance-response, whereby the first and the second concentration are different,
e) determining/calculating from the surface-plasmon-resonance-responses as determined in the previous steps the avidity-based binding strength of the at least bispecific binder to the first and the second antigen.

In one embodiment of all aspects and embodiments of the invention the at least bispecific binder is a bispecific antibody.

In one embodiment of all aspects and embodiments of the invention the first antigen is at least a fragment of the first antigen comprising the epitope of the first binding site of the bispecific binder and the second antigen is at least a fragment of the second antigen comprising the epitope of the second binding site of the bispecific binder.

In one embodiment of all aspects and embodiments of the invention the solid phase is a surface plasmon resonance chip.

One aspect of the invention is fusion-polypeptide of a first antigen and a second antigen of an at least bispecific binder.

Such a fusion protein according to the current invention is termed “first-antigen-second-antigen-fusion-polypeptide” herein.

In one embodiment of all aspects and embodiments of the invention the fusion-polypeptide according to the invention comprises as first antigen at least a fragment of the first antigen comprising the epitope of the first binding site of an at least bispecific binder and as second antigen at least a fragment of the second antigen comprising the epitope of the second binding site of an at least bispecific binder.

In one embodiment of all aspects and embodiments of the invention the fusion-polypeptide according to the invention is a linear polypeptide wherein the first antigen is at the C-terminus and the second antigen is at the N-terminus, or vice versa. In one embodiment the first antigen and the second antigen are connected by a peptidic linker. In one embodiment the peptidic linker comprises a tag for immobilization to a solid phase.

In one embodiment of all aspects and embodiments of the invention the fusion-polypeptide is a heterodimeric polypeptide comprising a first polypeptide, which is a fusion polypeptide of the first antigen and a first antibody heavy chain Fc-region polypeptide comprising a first set of heterodimerizing mutations, and a second polypeptide, which is a fusion polypeptide of the second antigen and a second antibody heavy chain Fc-region polypeptide comprising a second set of heterodimerizing mutations complementary to the first set of heterodimerizing mutations. In one embodiment the first antigen and the second antigen are at the N-terminus of the respective first or second Fc-region polypeptide. In one embodiment one or both of the Fc-region polypeptides comprise a tag for immobilization to a solid phase. In one embodiment the tag is at the C-terminus of the respective Fc-region polypeptide. In one embodiment the Fc-region is of the human IgG1 isotype. In one embodiment the first and the second set of heterodimerizing mutations are T366W and T366S/L368A/Y407V, respectively, or vice versa. In one embodiment the tag for immobilization is on histidine tag or a biotin.

In one embodiment of all aspects and embodiments of the invention the fusion-polypeptide is a heterodimeric polypeptide comprising a first polypeptide, which is a fusion polypeptide of the first antigen and a first antibody heavy chain constant region polypeptide comprising a first set of heterodimerizing mutations, and a second polypeptide, which is a fusion polypeptide of the second antigen and a second antibody heavy chain constant region polypeptide comprising a second set of heterodimerizing mutations complementary to the first set of heterodimerizing mutations. In one embodiment the first antigen and the second antigen are at the N-terminus of the respective first or second constant region polypeptide. In one embodiment one or both of the constant region polypeptides comprise a tag for immobilization to a solid phase. In one embodiment the tag is at the C-terminus of the respective Fc-region polypeptide. In one embodiment the Fc-region is of the human IgG1 isotype. In one embodiment the first and the second set of heterodimerizing mutations are T366W and T366S/L368A/Y407V, respectively, or vice versa. In one embodiment the tag for immobilization is on histidine tag or a biotin.

Thus, in general terms, one aspect of the current invention is a heterodimeric fusion polypeptide comprising
i) a first proteinaceous moiety, and
ii) a second proteinaceous moiety,
wherein
   the first proteinaceous moiety and the second proteinaceous moiety are the first and the second antigen of an at least bispecific antibody which comprises a first binding site that specifically binds to the first proteinaceous moiety and a second binding site that specifically binds to the second proteinaceous moiety,
   the first proteinaceous moiety is fused to the N-terminus of a first antibody heavy chain Fc-region polypeptide of the IgG1 subtype,
   the second proteinaceous moiety is fused to the N-terminus of a second antibody heavy chain Fc-region polypeptide of the IgG1 subtype,
   the first and the second heavy chain Fc-region polypeptide form a disulfide-linked heterodimer,
   one or both of the heavy chain Fc-region polypeptides comprise a tag for immobilization to a solid phase at its C-terminus, and
   the first and the second Fc-region polypeptide comprise the mutations T366W and T366S/L368A/Y407V, respectively.

Such a heterodimeric fusion polypeptide is a fusion-polypeptide of a first antigen and a second antigen of an at least bispecific binder.

In one embodiment of all aspects and embodiments of the invention the fusion-polypeptide according to the invention comprises only one, i.e. exactly one, first antigen and only one, i.e. exactly one, second antigen.

In one embodiment of all aspects and embodiments of the invention the first antigen and the second antigen are not the same polypeptide.

In one embodiment of all aspects and embodiments of the invention the first proteinaceous moiety and the second proteinaceous moiety are not from the same polypeptide.

One aspect of the invention is the use of the fusion-polypeptide of a first antigen and a second antigen of an at least bispecific binder according to the current invention in a surface-plasmon-resonance-method for the determination of the avidity-based binding strength of an at least bispecific binder, which comprises a first binding site specifically binding to a first antigen and a second binding site specifically binding to a second antigen, to said first and second antigen.

One aspect of the invention is an affinity chromatography column comprising the fusion-polypeptide of a first antigen and a second antigen of an at least bispecific binder according to the current invention as chromatography ligand.

One aspect of the invention is a method for separating/purifying an at least bispecific binder, which comprises a first binding site specifically binding to a first antigen and a second binding site specifically binding to a second antigen, with avidity-based binding to the first and second antigen from bispecific binder binding to the same first and second antigen without avidity-based binding, comprising the following steps:
a) applying a solution comprising at least bispecific binder with and without avidity-based binding to the first and second antigen to an affinity chromatography column according to the current invention,
b) recovering the at least bispecific binder with avidity-based binding to the first and second antigen from the column and thereby separating/purifying an at least bispecific binder from bispecific binder binding to the same first and second antigen without avidity-based binding.

One aspect of the invention is a method for purifying an at least bispecific binder, which comprises a first binding site specifically binding to a first antigen and a second binding site specifically binding to a second antigen, with avidity-based binding to the first and second antigen (from product- and/or process-related impurities), comprising the following steps:
a) applying a solution comprising the at least bispecific binder (and process- and/or product-related impurities) with avidity-based binding to the first and second antigen to an affinity chromatography column according to the current invention,
b) optionally washing the column whereby the at least bispecific binder with avidity-based binding to the first and second antigen remains bound to the column, and
c) recovering the at least bispecific binder with avidity-based binding to the first and second antigen from the column and thereby purifying a bispecific binder (from product- and/or process-related impurities).

A surface-plasmon-resonance-chip comprising the fusion-polypeptide of a first antigen and a second antigen of an at least bispecific binder according to the current invention immobilized in at least one flow cell.

A method for producing a surface-plasmon-resonance-chip comprising the fusion-polypeptide of a first antigen and a second antigen of an at least bispecific binder according to the current invention immobilized in at least one flow cell, comprising the following step
immobilizing either directly or via a specific binding pair the fusion-polypeptide of a first antigen and a second antigen of an at least bispecific binder according to the current invention in at least one flow cell of the surface-plasmon-resonance-chip.

One aspect of the invention is a method for determining the avidity-based binding strength of an at least bispecific binder, which comprises a first binding site specifically binding to a first antigen and a second binding site specifically binding to a second antigen, comprising the step of
determining the avidity-based binding strength of the at least bispecific binder from the change of the surface-plasmon-resonance-signal obtained by applying a solution comprising a first-antigen-second-antigen-fusion-polypeptide to a solid phase to which the bispecific binder is conjugated.

One aspect of the invention is a method for determining the avidity-based binding strength of an at least bispecific binder, which comprises a first binding site specifically binding to a first antigen and a second binding site specifically binding to a second antigen, to the first and second antigen, comprising the steps of
a) capturing the at least bispecific binder on a solid phase and determining/measuring/establishing a first surface-plasmon-resonance-response,
b) applying to the solid phase of step a) a solution comprising a first-antigen-second-antigen-fusion-polypeptide to form a captured first-antigen-second-antigen-fusion-polypeptide-bispecific-binder-complex and determining/measuring/establishing a second surface-plasmon-resonance-response,
c) determining/calculating from the difference between the first and the second surface-plasmon-resonance-response the avidity-based binding strength of the at least bispecific binder to the first and the second antigen.

One aspect of the invention is a method for determining the avidity-based binding strength of an at least bispecific binder, which comprises a first binding site specifically binding to a first antigen and a second binding site specifically binding to a second antigen, to the first and second antigen, comprising the steps of
a) capturing the at least bispecific binder on a solid phase (and determining/measuring/establishing a baseline surface-plasmon-resonance-response),
b) applying to the solid phase of step a) a first solution comprising a first-antigen-second-antigen-fusion-polypeptide at a first concentration to form a captured first-antigen-second-antigen-fusion-polypeptide-bispecific-binder-complex and determining/measuring/establishing a first surface-plasmon-resonance-response, (whereby the first surface-plasmon-resonance-response is the change of the surface-plasmon-resonance-response obtained by applying the first-antigen-second-antigen-fusion-polypeptide to the solid phase to the baseline surface-plasmon-resonance-response,)
c) dissociating the captured first-antigen-second-antigen-fusion-polypeptide-bispecific-binder-complex and thereby regenerating the solid phase,
d) repeating steps b) and c) at least with a second solution comprising the first-antigen-second-antigen-fusion-polypeptide at a second concentration and determining/measuring/establishing a second surface-plasmon-resonance-response, whereby all concentrations are different,
e) determining/calculating from the surface-plasmon-resonance-responses as determined in the previous steps the avidity-based binding strength of the at least bispecific binder to the first and the second antigen.

In one embodiment of all aspects and embodiments of the invention the at least bispecific binder is a bispecific antibody.

In one embodiment of all aspects and embodiments of the invention the first antigen is at least a fragment of the first antigen comprising the epitope of the first binding site of the at least bispecific binder and the second antigen is at least a fragment of the second antigen comprising the epitope of the second binding site of the at least bispecific binder.

In one embodiment of all aspects and embodiments of the invention the solid phase is a surface plasmon resonance chip.

### Quality Assessment of Samples Relative to a Reference

Antibodies, capable of avid binding will always bind to both specificities in this setting, as the local concentration of the second binding partner is massively increased after the initial first binding event, due to the close proximity. Since the avidity effect has a huge impact on the dissociation rate constant kd of the antibody to its antigens, it is possible to assess the relative activity which correlates with the antibodies potency, simply by reading out a single response value in the dissociation phase of an SPR measurement. As the kd influences the equilibrium concentration, ELISA or similar methods can also utilize this approach to assess the bispecific binding capability of an antibody to all of its targets.

Thus, one aspect according to the invention is a method for assessing the quality/purity/homogeneity of a sample comprising an at least bivalent, bispecific antibody comprising the following steps:
separately applying solutions comprising a covalent fusion polypeptide, which comprises at one terminus the first antigen and at a different, second terminus the second antigen of the bivalent, bispecific antibody, at different concentrations to an SPR chip on which the bivalent, bispecific antibody has been immobilized and monitoring the SPR-signal thereafter,
and
comparing the determined readout with the readout of a reference sample (obtained in the same way) and thereby determining the quality/purity/homogeneity of the sample comprising the at least bivalent, bispecific antibody,
wherein the at least bivalent, bispecific antibody comprises a first binding site specifically binding to a first, non-antibody antigen and a second binding site specifically binding to a second, different, non-antibody antigen.

In one embodiment the method comprises the following steps
separately applying solutions comprising a covalent fusion polypeptide, which comprises at one terminus the first antigen and at a different, second terminus the second antigen of the bivalent, bispecific antibody, at at least two different concentrations to an SPR chip on which the bivalent, bispecific antibody has been immobilized and monitoring the SPR-signal thereafter,
plotting the binding response (in resonance units) against the respective sample concentration,
fitting the data points of the obtained plot using a 2-parametric line fit and determining the y-axis intercept as readout,
comparing the determined readout by parallel-line transformation with that of a reference sample (analyzed in the same way) and thereby determining the quality/purity/homogeneity of the sample comprising the at least bivalent, bispecific antibody,
wherein the at least bivalent, bispecific antibody comprises a first binding site specifically binding to a first, non-antibody antigen and a second binding site specifically binding to a second, different, non-antibody antigen.

The method is based on an SPR assay.

In the first step, an antibody specifically binding to the antibody in question, e.g. if the antibody in question comprises the mutations LALA (L234A/L235A) and PG (P329G) in its Fc-region an antibody specifically binding to the LALA mutation or the PG mutation in the Fc-region of the sample antibody, is immobilized to an SPR sensor chip according to the manufacturer's instructions. In this example a bivalent, bispecific antibody in domain exchange format having the mutations P329G and L234A/L235A in the Fc-region is used (denoted as LALA PG herein; numbering according to Kabat). Likewise any other mutation in the Fc-region can be used as long as a capture reagent specifically binding thereto is available.

At least 16,000 RU (“Response Bound”) should be immobilized to ensure that antigen capturing is not limited by the immobilization. A reference control flow cell is treated in the same way. Finally, both surfaces are blocked. A preferred immobilization buffer is HBS-EP+ (10 mM HEPES, 150 mM NaCl pH 7.4, GE Healthcare).

Second, the bivalent, bispecific antibody is injected.

Third, an antigen 1-antigen 2-Fc-region-fusion according to the current invention is injected on the second flow cell at different concentrations.

SPR-signals are detected. The binding responses (resonance units, RU) of the antigen 1-antigen 2-Fc-region-fusion correlate with the amount of bivalent, bispecific antibody and is plotted against the sample concentration range used. The resulting linear plots are analyzed by appropriate computer software (e.g. XLfit4, IDBS Software), which fits a 2-parametric line and hence allows determination of the y-axis intercept, which is equivalent to the biological binding activity (=potency) readout. Using, e.g., a parallel line-transformation, the relative potency of a sample in comparison to the antibody reference standard can be determined (=reportable potency).

Antigen 1 and antigen 2 bind to the captured bivalent, bispecific antibody simultaneously. The targets binding response is used as final assay readout.

The method described above is designed to fulfill the criteria of the USP 1032 for potency release assays, as published by Gassner et al. (Gassner, C., et al. J. Pharm. Biomed. Anal. 102(2015 ) 144-149). However, measuring a single concentration and plotting it against a calibration curve of a reference standard is sufficient if there is no need to fulfill the USP 1032 criteria.

In an alternative setting, one aspect according to the invention is a method for assessing the quality/purity/homogeneity of a sample comprising an at least bivalent, bispecific antibody comprising the following steps:
separately applying solutions comprising the bivalent, bispecific antibody at different concentrations to an SPR chip on which a covalent fusion polypeptide, which comprises at one terminus the first antigen and at a different, second terminus the second antigen of the bivalent, bispecific antibody, has been immobilized and monitoring the SPR-signal thereafter,
and
comparing the determined readout with the readout of a reference sample (obtained in the same way) and thereby determining the quality/purity/homogeneity of the sample comprising the at least bivalent, bispecific antibody,
wherein the at least bivalent, bispecific antibody comprises a first binding site specifically binding to a first, non-antibody antigen and a second binding site specifically binding to a second, different, non-antibody antigen.

In one embodiment the method comprises the following steps
separately applying solutions comprising the bivalent, bispecific antibody at at least two different concentrations to an SPR chip on which a covalent fusion polypeptide, which comprises at one terminus the first antigen and at a different, second terminus the second antigen of the bivalent, bispecific antibody, has been immobilized and monitoring the SPR-signal thereafter,
plotting the binding response (in resonance units) against the respective sample concentration,
fitting the data points of the obtained plot using a 2-parametric line fit and determining the y-axis intercept as readout,
comparing the determined readout by parallel-line transformation with that of a reference sample (analyzed in the same way) and thereby determining the quality/purity/homogeneity of the sample comprising the at least bivalent, bispecific antibody,
wherein the at least bivalent, bispecific antibody comprises a first binding site specifically binding to a first, non-antibody antigen and a second binding site specifically binding to a second, different, non-antibody antigen.

The method is based on an SPR assay.

In the first step, an antibody specifically binding to the antigen 1-antigen 2-Fc-region fusion, e.g. if the antigen 1-antigen 2-Fc-region-fusion comprises the mutations LALA (L234A/L235A) and PG (P329G) in its Fc-region an antibody specifically binding to the LALA mutation or the PG mutation in the Fc-region of the sample antibody, is immobilized to an SPR sensor chip according to the manufacturer's instructions. At least 16,000 RU (“Response Bound”) should be immobilized to ensure that antibody capturing is not limited by the immobilization. A reference control flow cell is treated in the same way. Finally, both surfaces are blocked. A preferred immobilization buffer is HBS-EP+ (10 mM HEPES, 150 mM NaCl pH 7.4, GE Healthcare).

Second, an antigen 1-antigen 2-Fc-region-fusion according to the current invention is injected.

Third, the bivalent, bispecific antibody is injected at different concentrations.

SPR-signals are detected. The binding responses (resonance units, RU) of bivalent, bispecific antibody correlate with the amount of the antigen 1-antigen 2-Fc-region-fusion and is plotted against the sample concentration range used. The resulting linear plots are analyzed by appropriate computer software (e.g. XLfit4, IDBS Software), which fits a 2-parametric line and hence allows determination of the y-axis intercept, which is equivalent to the biological binding activity (=potency) readout. Using, e.g., a parallel line-transformation, the relative potency of a sample in comparison to the antibody reference standard can be determined (=reportable potency).

Captured antigen 1 and antigen 2 bind to the bivalent, bispecific antibody simultaneously. The targets binding response is used as final assay readout.

The method described above is designed to fulfill the criteria of the USP 1032 for potency release assays, as published by Gassner et al. (Gassner, C., et al. J. Pharm. Biomed. Anal. 102 (2015 ) 144-149). However, measuring a single concentration and plotting it against a calibration curve of a reference standard is sufficient if there is no need to fulfill the USP 1032 criteria.

### SUMMARY

At least a part of the current invention relates to:
1. A method for determining the avidity-based binding strength of an at least bivalent, bispecific antibody to its first and second antigen, the method comprising
   determining the avidity-based binding strength of the bivalent, bispecific antibody from the surface-plasmon-resonance signal obtained by applying a solution comprising the bivalent, bispecific antibody to a solid phase to which a covalent fusion polypeptide, which comprises at one terminus the first antigen and at a different, second terminus the second antigen, is conjugated and monitoring the SPR-signal thereafter,
   wherein the at least bivalent, bispecific antibody comprises a first binding site specifically binding to a first, non-antibody antigen and a second binding site specifically binding to a second, different, non-antibody antigen.
2. The method according to item 1 comprising the following steps:
   a) capturing a first-antigen-second-antigen-fusion-polypeptide on a solid phase,
   b) applying to the solid phase of step a) a first solution comprising the bivalent, bispecific antibody at a first concentration to form a captured first-antigen-second-antigen-fusion-polypeptide-bivalent, bispecific-antibody-complex and determining a first surface-plasmon-resonance-response,
   c) dissociating the captured first-antigen-second-antigen-fusion-polypeptide-bivalent, bispecific-antibody-complex and thereby regenerating the solid phase,
   d) repeating steps b) and c) at least with a second solution comprising the bivalent, bispecific antibody at a second concentration and determining a second surface-plasmon-resonance-response, whereby all concentrations are different,
   e) determining from the surface-plasmon-resonance-responses as determined in the previous steps the avidity-based binding strength of at least the bivalent, bispecific antibody to the first and the second antigen.
3. The method according to any one of items 1 to 2, wherein each covalent fusion polypeptide, which comprises at one terminus the first antigen and at a different, second terminus the second antigen, is conjugated to the solid phase separately.
4. The method according to any one of items 1 to 3, wherein the first antigen is at least a fragment of the first antigen comprising the epitope of the first binding site of the bivalent, bispecific antibody and the second antigen is at least a fragment of the second antigen comprising the epitope of the second binding site of the bivalent, bispecific antibody.
5. The method according to any one of items 1 to 4, wherein the first antigen is different from the second antigen.
6. The method according to any one of items 1 to 5, wherein the solid phase is a surface plasmon resonance chip.
7. The method according to any one of items 1 to 6, wherein the first-antigen-second-antigen-fusion-polypeptide is a heterodimeric polypeptide comprising a first polypeptide, which is a fusion polypeptide of the first antigen and a first antibody heavy chain Fc-region polypeptide comprising a first set of heterodimerizing mutations, and a second polypeptide, which is a fusion polypeptide of the second antigen and a second antibody heavy chain Fc-region polypeptide comprising a second set of heterodimerizing mutations complementary to the first set of heterodimerizing mutations.
8. The method according to item 7, wherein the first antigen and the second antigen are at the N-terminus of the respective first or second Fc-region polypeptide.
9. The method according to any one of items 1 to 8, wherein the first-antigen-second-antigen-fusion-polypeptide comprise a tag for immobilization to a solid phase.
10. The method according to item 9, wherein the tag is at the C-terminus of the respective Fc-region polypeptide.
11. The method according to any one of items 7 to 10, wherein the Fc-region is of the human IgG1 isotype.
12. The method according to any one of items 7 to 11, wherein the first and the second set of heterodimerizing mutations are T366W and T366S/L368A/Y407V, respectively, or vice versa.
13. A heterodimeric fusion polypeptide comprising
   i) a first polypeptide, and
   ii) a second polypeptide,
   wherein
      the first polypeptide and the second polypeptide are the first and the second antigen of a bispecific antibody which comprises a first binding site that specifically binds to the first polypeptide and a second binding site that specifically binds to the second polypeptide,
      the first polypeptide is fused to the N-terminus of a first antibody heavy chain Fc-region polypeptide of the IgG1 subtype,
      the second polypeptide is fused to the N-terminus of a second antibody heavy chain Fc-region polypeptide of the IgG1 subtype,
      the first and the second heavy chain Fc-region polypeptide form a disulfide-linked heterodimer,
      one or both of the heavy chain Fc-region polypeptides comprise a tag for immobilization to a solid phase at its C-terminus,
      the first and the second Fc-region polypeptide comprise the mutations T366W and T366S/L368A/Y407V, respectively, and
      the first antigen is different from the second antigen.
14. The use of a heterodimeric fusion polypeptide according to item 13 for the determination of the avidity-based binding strength of a bispecific antibody, which comprises a first binding site specifically binding to a first antigen and a second binding site specifically binding to a second antigen, to said first and second antigen in a surface-plasmon-resonance-method.
15. A method for purifying a bispecific antibody, which comprises a first binding site specifically binding to a first antigen and a second binding site specifically binding to a second antigen, with avidity-based binding to the first and second antigen from product- and/or process-related impurities, comprising the following steps:
   a) applying a solution comprising the bispecific antibody with avidity-based binding to the first and second antigen and process- and/or product-related impurities to an affinity chromatography column comprising the heterodimeric fusion polypeptide according to item 13 as chromatography ligand,
   b) optionally washing the column whereby the bispecific antibody with avidity-based binding to the first and second antigen remains bound to the column, and
   c) recovering the bispecific antibody with avidity-based binding to the first and second antigen from the column and thereby purifying a bispecific antibody (from product- and/or process-related impurities).

The following examples and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1: Scheme of affinity-based binding and avidity-based binding.

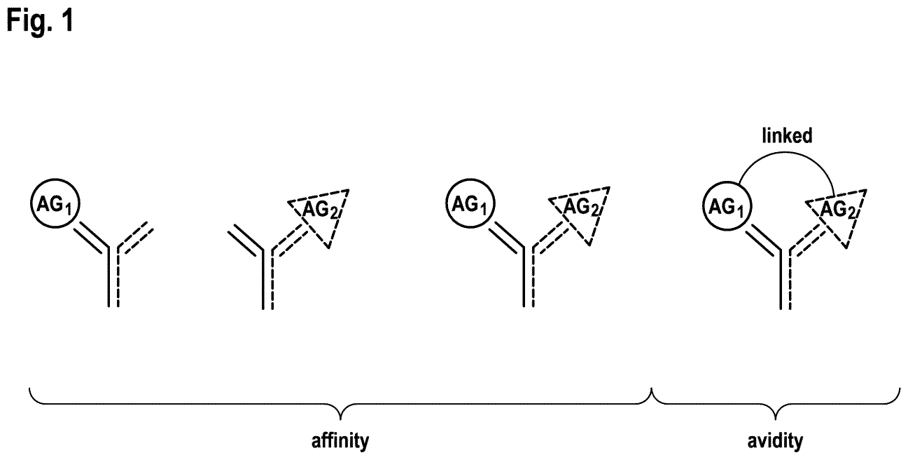

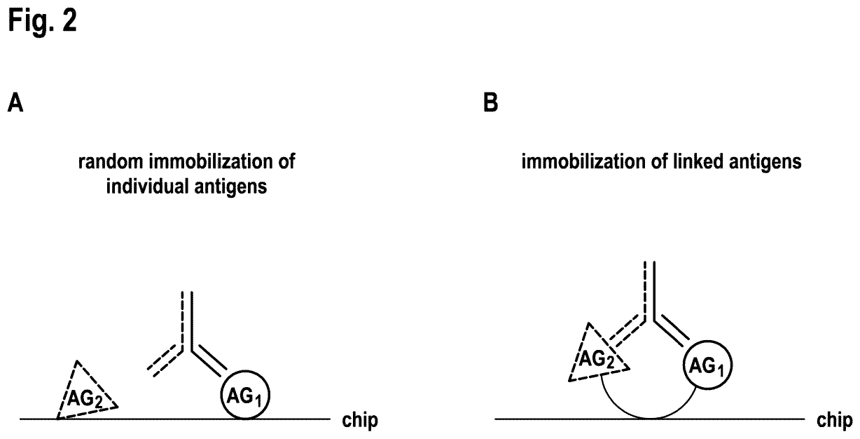

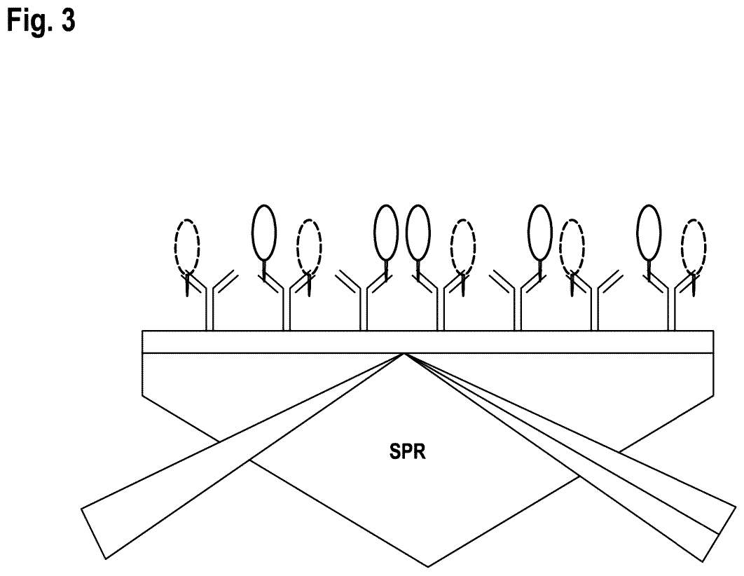

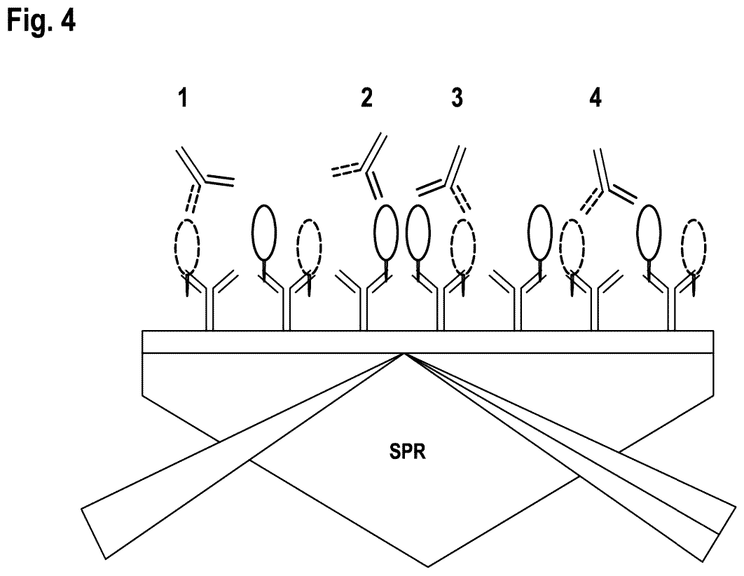

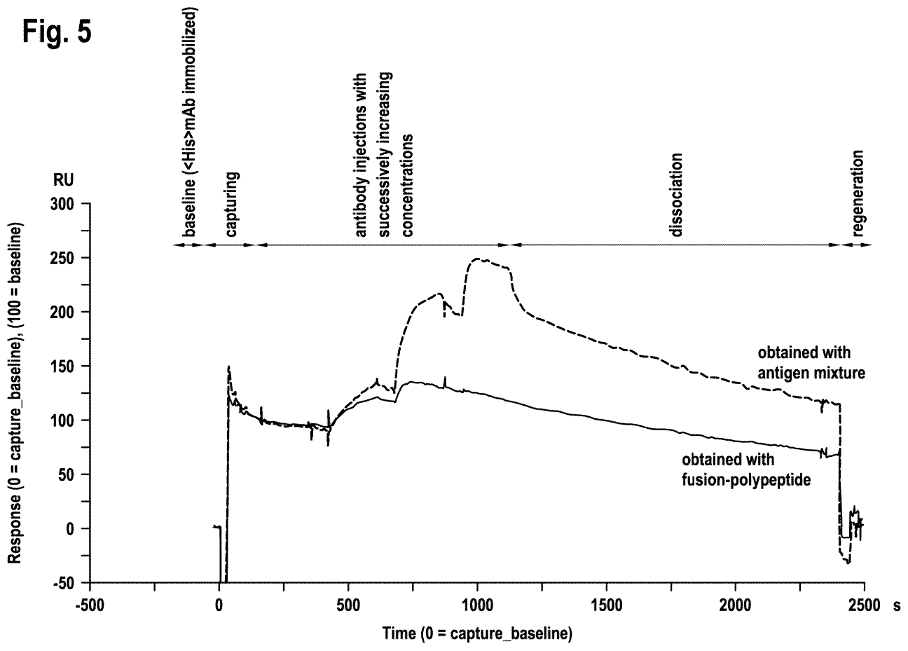

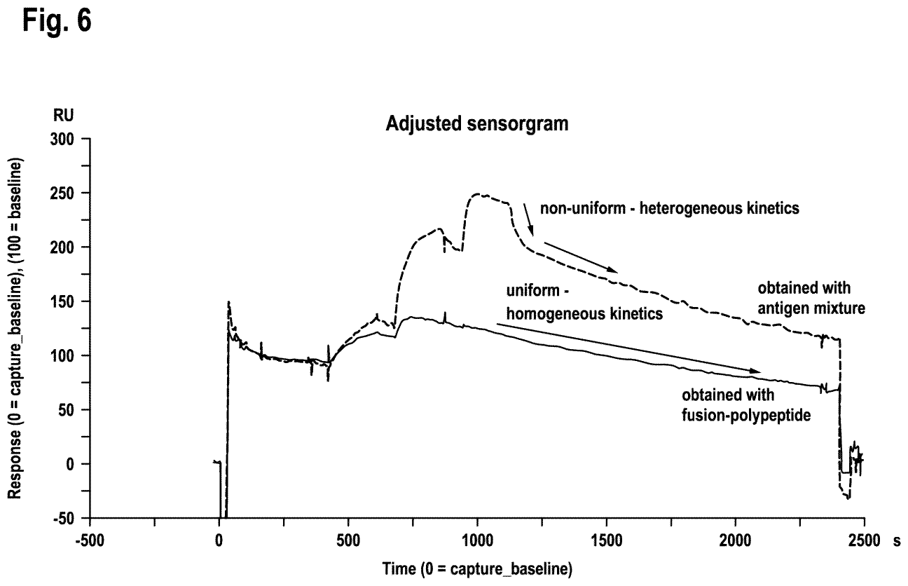

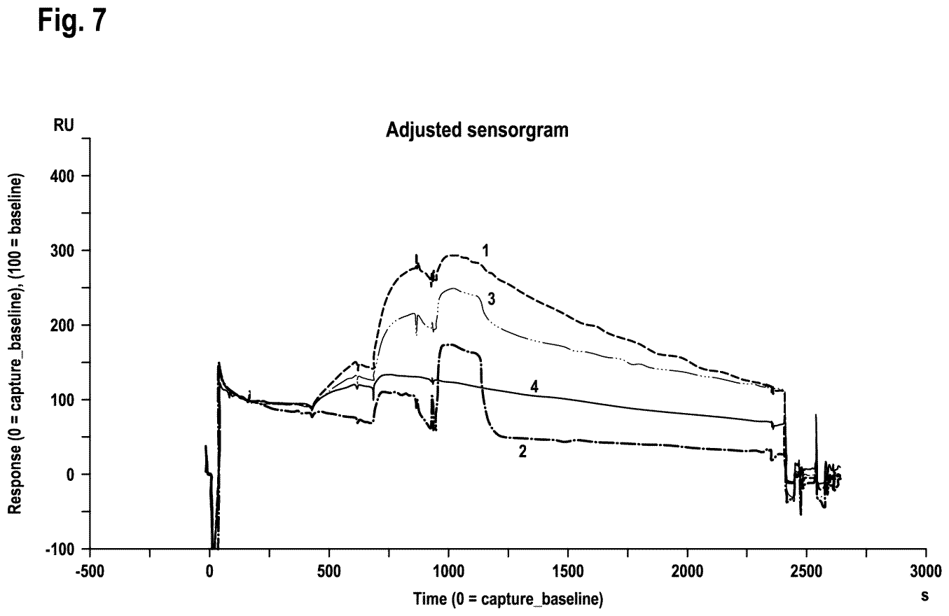

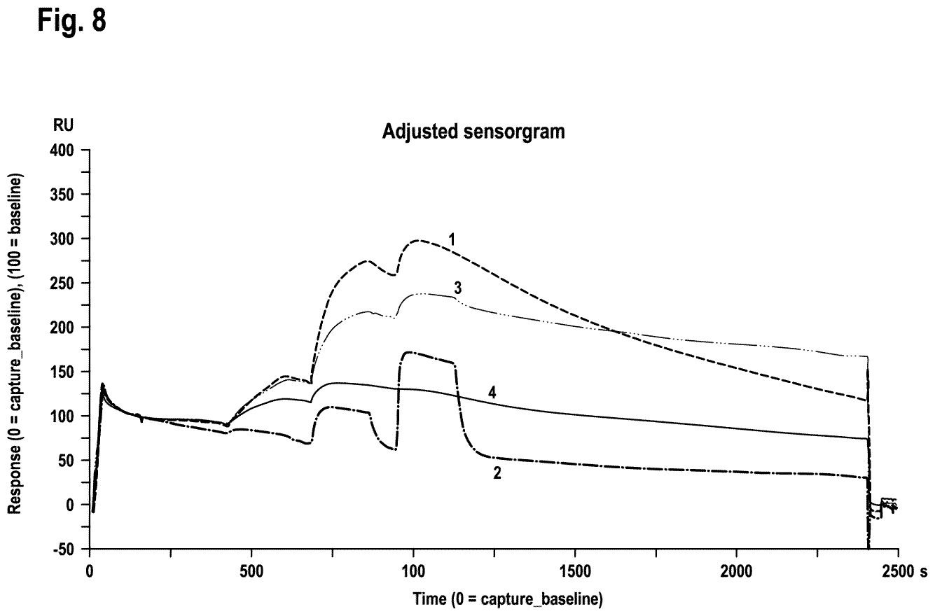

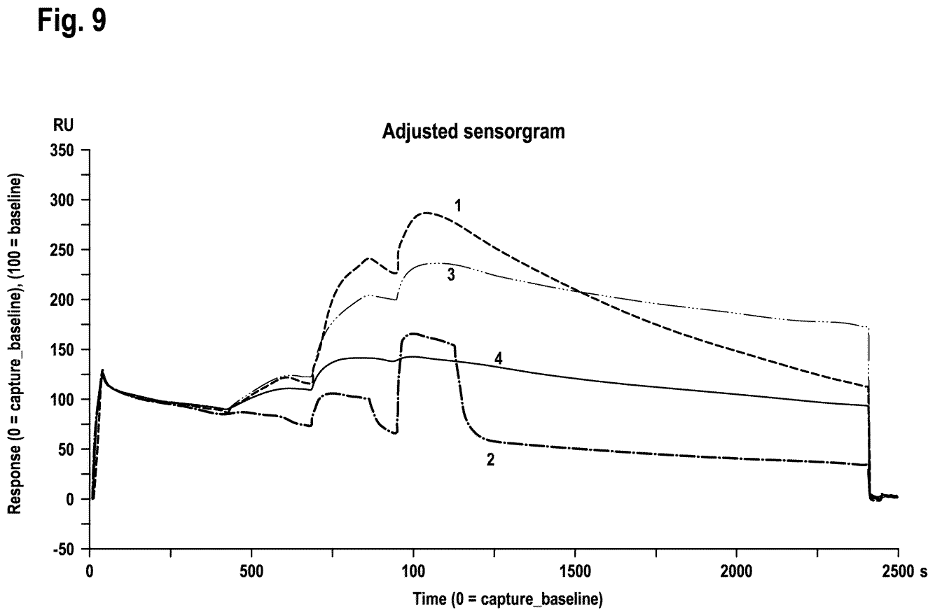

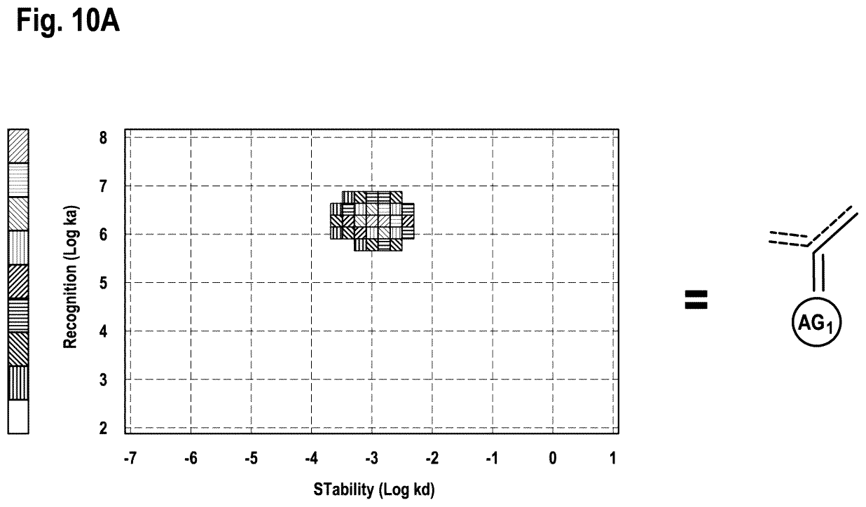

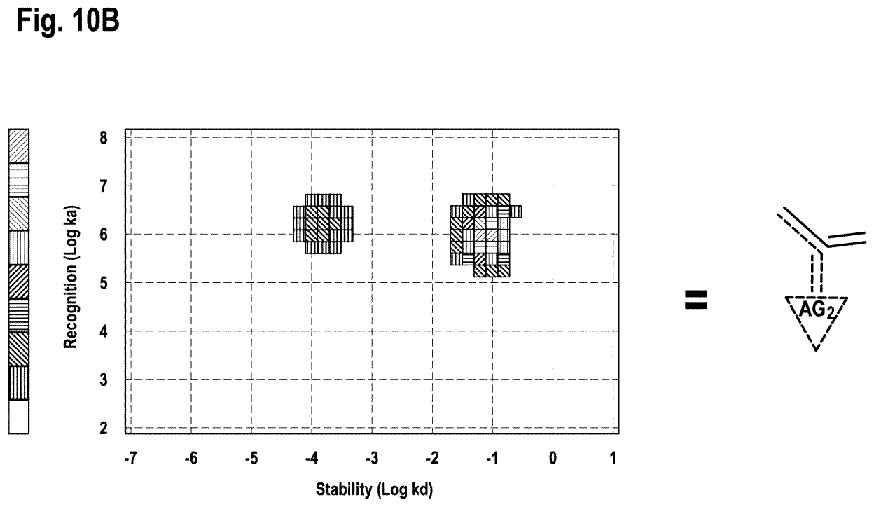

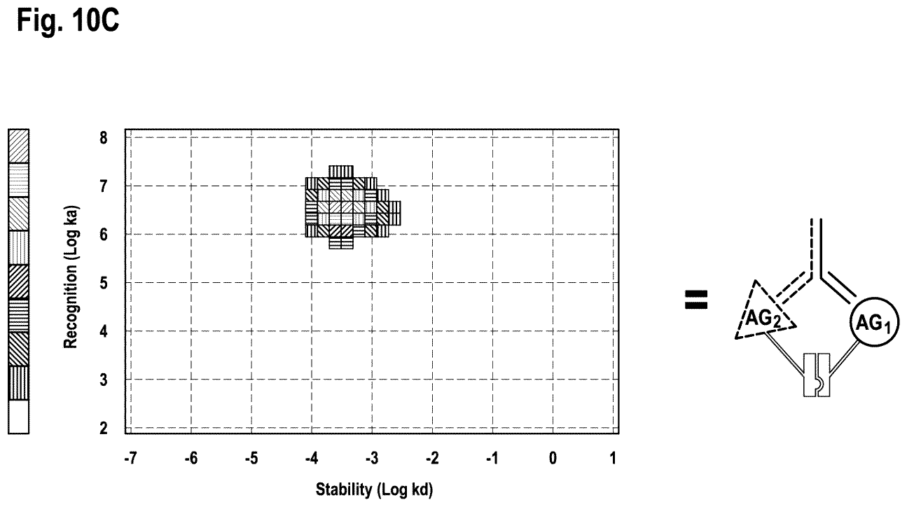

FIG. 2: A: SPR setup with random immobilization of antigen mixtures. In SPR setups with random immobilization of individual antigens, i.e. which have an uneven distribution of the different antigens, antibodies that bind only to one antigen will be present since the two antigens are too far apart resulting in the determination of a mixture of affine and avid binding.
B: SPR setup with defined immobilization of linked antigens. In SPR setups with defined immobilization of linked antigens, i.e. which have an even distribution of the different antigens, the antibodies will bind to both antigens simultaneously since the two antigens are properly spaced apart resulting in the determination of avid binding.

FIG. 3: Scheme of the surface of an SPR chip obtained by immobilizing unlinked antigens. For capture each antigen comprises a tag and is captured using an anti-tag antibody. Thereby a random surface is generated comprising a mixture of anti-tag antibody either only with the first antigen or only with the second antigen or with both antigens. Thus, no homogeneous surface but an uneven distribution of the antigens on the surface is obtained.

FIG. 4: Different binding modes of a bispecific antibody resulting from the uneven distribution of the antigens on an SPR chip surface:
1 and 2: binding with one binding site only;
3: simultaneous binding with both binding sites to a single complex;
4: simultaneous binding with both binding sites to two different complexes.

FIG. 5: SPR sensograms overlay of two sensograms obtained under identical conditions with the same bispecific antibody differing only in the format of the antigens captured on the chip surface:
upper sensogram: obtained with antigen mixture;
lower sensogram: obtained with fusion polypeptide according to the current invention;
Only in the sensogram obtained with a method according to the current invention a homogeneous and analyzable response curve is obtained.
Immobilization level 5-15 RU.

FIG. 6: Same sensograms as in FIG. 5 with additional annotations.

FIG. 7: SPR sensograms overlay of four sensograms obtained under identical conditions with the same bispecific antibody differing only in the format of the antigens captured on the chip surface:
1: antigen 1 only;
2: antigen 2 only;
3: antigen mixture;
4: fusion polypeptide according to the current invention;
Only in the sensogram obtained with a method according to the current invention a homogeneous and analyzable response curve is obtained.
(same experimental conditions as for FIG. 5)

FIG. 8: Same as FIG. 7 but obtained at 50-120 RU immobilization level.
1: antigen 1 only;
2: antigen 2 only;
3: antigen mixture;
4: fusion polypeptide according to the current invention;
Only in the sensogram obtained with a method according to the current invention a homogeneous and analyzable response curve is obtained.

FIG. 9: Same as FIG. 7 but obtained at 300-600 RU immobilization level.
1: antigen 1 only;
2: antigen 2 only;
3: antigen mixture;
4: fusion polypeptide according to the current invention;
Only in the sensogram obtained with a method according to the current invention a homogeneous and analyzable response curve is obtained.

FIG. 10A-C: Kinetic rate maps for the interaction of
FIG. 10A: antigen 1 only;
FIG. 10B: antigen 2 only;
FIG. 10C: fusion polypeptide according to the current invention; with the same bispecific antibody.

### EXAMPLES

### Equipment and Reagents

All SPR experiments were performed on a BIAcore T200 instrument (GE Healthcare) at 25 °C. Antibodies were manufactured by Roche Diagnostics GmbH, Mannheim, Germany, if not mentioned otherwise.

### Recombinant DNA Techniques

Standard methods were used to manipulate DNA as described in Sambrook, J. et al., Molecular Cloning: A laboratory manual; Cold Spring Harbor Laboratory Press, Cold Spring Harbor, New York, 1989. The molecular biological reagents were used according to the manufacturer's instructions.

### DNA Sequence Determination

DNA sequences were determined by double strand sequencing performed at MediGenomix GmbH (Martinsried, Germany) or SequiServe GmbH (Vaterstetten, Germany).

### EXAMPLE 1

### Expression Vectors, Expression and Purification

### Expression Vectors

For the expression of the described polypeptides/antibodies, expression plasmids for transient expression (e.g. in HEK293) were applied.

Beside the antibody/polypeptide expression cassette the vectors contained:
an origin of replication which allows replication of this plasmid in E. coli, and
a ß-lactamase gene which confers ampicillin resistance in E. coli.

The transcription unit of the antibody gene was composed of the following elements:
unique restriction site(s) at the 5′ end
the immediate early enhancer and promoter from the human cytomegalovirus,
followed by the Intron A sequence,
a 5′-untranslated region of a human antibody gene,
an immunoglobulin heavy chain signal sequence,
the respective antibody chain encoding nucleic acid
a 3′ untranslated region with a polyadenylation signal sequence, and
unique restriction site(s) at the 3′ end.

The fusion genes encoding the antibodies and fusion polypeptides as described herein were generated by PCR and/or gene synthesis and assembled by known recombinant methods and techniques by connection of the according nucleic acid segments e.g. using unique restriction sites in the respective vectors. The subcloned nucleic acid sequences were verified by DNA sequencing. For transient transfections larger quantities of the plasmids were prepared by plasmid preparation from transformed E. coli cultures (Nucleobond AX, Macherey-Nagel).

### Cell Culture Techniques

Standard cell culture techniques were used as described in Current Protocols in Cell Biology (2000), Bonifacino, J. S., Dasso, M., Harford, J. B., Lippincott-Schwartz, J. and Yamada, K. M. (eds.), John Wiley & Sons, Inc.

Bispecific antibodies and fusion polypeptides were expressed by transient co-transfection of the respective expression plasmids in HEK293-F cells growing in suspension as described below.

### Transient Transfections in Hek293 System

All bispecific antibodies and fusion polypeptides were generated by transient transfection of 293F cells using the Freestyle system (ThermoFisher). Here the 293F cells were cultivated in F17 Medium, transfected with 293Free (Novagen) and fed after 4 hours with VPA 4 mM and Feed 7 and 0.6% Glucose after 16 h. Further the Expi293F™ Expression System Kit (ThermoFisher) was used. Here the Expi293F™ cells were cultivated in Expi293™ Expression Medium and transfected using ExpiFectamine™ 293 Transfection Kit according manufacturer's instructions. Cell supernatants were harvested after 7 days and purified by standard methods.

### Protein Determination

The protein concentration of purified antibodies and derivatives was determined by determining the optical density (OD) at 280 nm, using the molar extinction coefficient calculated on the basis of the amino acid sequence according to Pace, et al., Protein Science, 1995, 4, 2411-1423.

### Antibody Concentration Determination in Supernatants

The concentration of antibodies in cell culture supernatants was estimated by immunoprecipitation with Protein A Agarose-beads (Roche). 60 μL Protein A Agarose beads were washed three times in TBS-NP 40 (50 mM Tris, pH 7.5, 150 mM NaCl, 1% Nonident-P40). Subsequently, 1-15 mL cell culture supernatant was applied to the Protein A Agarose beads pre-equilibrated in TBS-NP40. After incubation for at 1 hour at room temperature the beads were washed on an Ultrafree-MC-filter column (Amicon) once with 0.5 mL TBS-NP 40, twice with 0.5 mL 2× phosphate buffered saline (2× PBS, Roche) and briefly four times with 0.5 mL 100 mM Na-citrate pH 5.0. Bound antibody was eluted by addition of 35 μl NuPAGE® LDS Sample Buffer (Invitrogen). Half of the sample was combined with NuPAGE® Sample Reducing Agent or left unreduced, respectively, and heated for 10 min at 70 °C. Consequently, 5-30 μl were applied to a 4-12% NuPAGE® Bis-Tris SDS-PAGE (Invitrogen) (with MOPS buffer for non-reduced SDS-PAGE and MES buffer with NuPAGE® Antioxidant running buffer additive (Invitrogen) for reduced SDS-PAGE) and stained with Coomassie Blue.

The concentration of antibodies and derivatives in cell culture supernatants was quantitatively measured by affinity HPLC chromatography. Briefly, cell culture supernatants containing antibodies and derivatives that bind to Protein A were applied to an Applied Biosystems Poros A/20 column in 200 mM KH2PO4, 100 mM sodium citrate, pH 7.4 and eluted from the matrix with 200 mM NaCl, 100 mM citric acid, pH 2,5 on an Agilent HPLC 1100 system. The eluted protein was quantified by UV absorbance and integration of peak areas. A purified standard IgG1 antibody served as a standard.

Alternatively, the concentration of antibodies and derivatives in cell culture supernatants was measured by Sandwich-IgG-ELISA. Briefly, StreptaWell High Bind Streptavidin A-96 well microtiter plates (Roche) are coated with 100 μL/well biotinylated anti-human IgG capture molecule F(ab′) 2<h-Fcγ> BI (Dianova) at 0.1 μg/mL for 1 hour at room temperature or alternatively overnight at 4 °C and subsequently washed three times with 200 μL/well PBS, 0.05% Tween (PBST, Sigma). 100 μL/well of a dilution series in PBS (Sigma) of the respective antibody containing cell culture supernatants was added to the wells and incubated for 1-2 hour on a microtiterplate shaker at room temperature. The wells were washed three times with 200 μL/well PBST and bound antibody was detected with 100 μl F(ab′)2<hFcγ>POD (Dianova) at 0.1 μg/mL as the detection antibody for 1-2 hours on a microtiterplate shaker at room temperature. Unbound detection antibody was washed away three times with 200 μL/well PBST and the bound detection antibody was detected by addition of 100 μL ABTS/well. Determination of absorbance was performed on a Tecan Fluor Spectrometer at a measurement wavelength of 405 nm (reference wavelength 492 nm).

### Protein Purification

Proteins were purified from filtered cell culture supernatants referring to standard protocols. In brief, antibodies were applied to a Protein A Sepharose column (GE healthcare) and washed with PBS. Elution of antibodies was achieved at pH 2.8 followed by immediate neutralization of the sample. Aggregated protein was separated from monomeric antibodies by size exclusion chromatography (Superdex 200, GE Healthcare) in PBS or in 20 mM Histidine, 150 mM NaCl pH 6.0. Monomeric antibody fractions were pooled, concentrated (if required) using e.g., a MILLIPORE Amicon Ultra (30 MWCO) centrifugal concentrator, frozen and stored at −20 °C or −80 °C. Part of the samples were provided for subsequent protein analytics and analytical characterization e.g. by SDS-PAGE, size exclusion chromatography (SEC) or mass spectrometry.

### SDS-PAGE

The NuPAGE® Pre-Cast gel system (Invitrogen) was used according to the manufacturer's instruction. In particular, 10% or 4-12% NuPAGE® Novex® Bis-TRIS Pre-Cast gels (pH 6.4) and a NuPAGE® MES (reduced gels, with NuPAGE® Antioxidant running buffer additive) or MOPS (non-reduced gels) running buffer was used.

### EXAMPLE 2

### Assay Procedure

A BIAcore T200 CM5 sensorchip was prepared by immobilizing about 3000-5000 RU of an anti-HIS antibody (GE Healthcare: His capture kit; No. 28995056) on flow cell three and four using standard amine coupling (GE Healthcare: amine coupling kit, type 1; No. BR100050) according to the manufacturer's instructions.

All sample- and preceding startup-cycles consisted of four commands:
1. Capture: After a 10 sec. stabilization period, a variable solution was injected into the second flow cell for 30 sec. at a flow rate of 10 μl/min.
2. Sample: A single cycle kinetic consisting of four different concentrations (0.5 nM, 5 nM, 50 nM, 500 nM) was injected to flow cell three and four at a flow rate of 30 μl/min. Association and dissociation times were set to 180 sec. and 1200 sec.
3. Regeneration: 100 mM Glycine-HCl at pH 1.5 was injected to both flow cells for 40 sec. at a flow rate of 30 μl/min.
4. Regeneration: Same settings as the previous command plus a 10 sec. long stabilization period.

A sample set consisting of the four different antigens 1) monomeric huAG1-His-tag, 2) monomeric huAG2-His-tag, 3) a mixture of monomeric huAG1-His-tag and monomeric huAG2-His-Tag, and 4) huAG1-huAG2-bispecific-Fc-fusion-His-tag was used at varying concentrations of 10 nM, 100 nM and 1000 nM in the capturing step to generate the sensor surface.

Afterwards, a bispecific anti-AG1/AG2 antibody concentration series was injected and allowed to bind to the single antigens, the antigen mixture, as well as the antigen fusion in subsequent measurement cycles. Always accompanied by a respective blank cycle for later referencing.

The resulting data was then processed using the BIAcore T200 Evaluation Software. If possible, 1:1 Langmuir fits were created.

### EXAMPLE 3

### Quality Assessment of Samples Relative to a Reference Standard

The method according to the current invention is based on an SPR assay exemplified in this Example using a BIAcore instrument (GE Healthcare). Any other instrument can likewise be used.

In this example a bivalent, bispecific antibody in domain exchange format having the mutations P329G and L234A/L235A in the Fc-region is used (denoted as LALA PG herein; numbering according to Kabat). Likewise any other mutation in the Fc-region can be used as long as a capture reagent specifically binding thereto is available.

In a first step, an antibody targeting the either the L234A/L235A mutation or the P329G mutation in the Fc-region is immobilized to a CM5 sensor chip using the provided amine coupling chemistry from GE Healthcare. Flow cells were activated with a 1:1 mixture of 0.4 M 1-ethyl-3-(3-dimethylaminopropyl)-carbodiimide (EDC) and 0.1 MN-hydroxysuccinimide (NHS) at a flow rate of 5 μl/min. Anti-LALA antibody or anti-PG antibody was injected in sodium acetate, pH 5.0 at 50 μg/ml for 1200 s, which resulted in a surface density of approximately 18,000 RU. At least 16,000 RU (“Response Bound”) should be immobilized to ensure that antibody capturing is not limited by the immobilization. A reference control flow cell was treated in the same way as described before. Finally, both surfaces were blocked with an injection of 1 M ethanolamine/HCl pH 8.5. As immobilization buffer HBS-EP+ (10 mM HEPES, 150 mM NaCl pH 7.4, GE Healthcare) was used.

Second, the bivalent, bispecific antibody in domain exchange format comprising the LALA PG mutation in the Fc-region was diluted in HBS-EP+ (GE Healthcare) and injected for 60 sec. at a flow rate of 5 μl/min.

Third, an antigen 1-antigen 2-Fc-fusion according to the invention was injected for 60 sec. with a concentration of 15 μg/ml. The dissociation time (washing with running buffer) was 600 s at a flow rate of 30 μl/min. All interactions were performed at 25 °C

Fourth, a regeneration solution of 10 mM NaOH was injected twice for 30 s at 30 μl/min flow to remove any non-covalently bound protein after each binding cycle followed by a stabilization period of 40 s to stabilize the baseline.

Signals were detected at a rate of one signal per second. The binding responses (resonance units, RU) of the antigen 1-antigen 2-Fc-fusion correlate with the amount of bivalent, bispecific antibody and are plotted against the sample concentration range used. The resulting linear plots were analyzed by appropriate computer software (e.g. XLfit4, IDBS Software), which fits a 2-parametric line and hence allows determination of the y-axis intercept as the biological binding activity (=potency) readout. Following a parallel line-transformation, the relative potency of a sample in comparison to the antibody reference standard can be reported (=reportable potency).

Antigen 1 and antigen 2 bind to the captured bivalent, bispecific antibody simultaneously. The targets binding response is used as final assay readout.

The method described above is designed to fulfill the criteria of the USP 1032 for potency release assays, as published by Gassner et al. (Gassner, C., et al. J. Pharm. Biomed. Anal. 102 (2015 ) 144-149). However, measuring a single concentration and plotting it against a calibration curve of a reference standard is sufficient if there is no need to fulfill the USP 1032 criteria.

### EXAMPLE 4

### Quality Assessment of Samples Relative to a Reference Standard

The method according to the current invention is based on an SPR assay exemplified in this Example using a BIAcore instrument (GE Healthcare). Any other instrument can likewise be used.

In this example an antigen 1-antigen 2-Fc-fusion having the mutations P 329G and L234A/L235A in the Fc-region is used (denoted as LALA PG herein; numbering according to Kabat). Likewise any other mutation in the Fc-region can be used as long as a capture reagent specifically binding thereto is available.

In a first step, an antibody targeting either the L234A/L235A mutation or the P329G mutation in the Fc-region of the antigen 1-antigen 2-Fc-fusion is immobilized to a CM5 sensor chip using the provided amine coupling chemistry from GE Healthcare. Flow cells were activated with a 1:1 mixture of 0.4 M 1-ethyl-3-(3-dimethylaminopropyl)-carbodiimide (EDC) and 0.1 MN-hydroxysuccinimide (NHS) at a flow rate of 5 μl/min. Anti-LALA antibody or anti-PG antibody was injected in sodium acetate, pH 5.0 at 50 μg/ml for 1200 s, which resulted in a surface density of approximately 18,000 RU. At least 16,000 RU (“Response Bound”) should be immobilized to ensure that antibody capturing is not limited by the immobilization. A reference control flow cell was treated in the same way as described before. Finally, both surfaces were blocked with an injection of 1 M ethanolamine/HCl pH 8.5. As immobilization buffer HBS-EP+ (10 mM HEPES, 150 mM NaCl pH 7.4, GE Healthcare) was used.

Second, an antigen 1-antigen 2-Fc-fusion according to the invention was injected for 60 sec. at a flow rate of 5 μl/min.

Third, a bivalent, bispecific antibody in domain exchange format was diluted in HBS-EP+ (GE Healthcare) and injected at different concentrations for 60 sec. with a concentration of 15 μg/ml. The dissociation time (washing with running buffer) was 600 s at a flow rate of 30 μl/min. All interactions were performed at 25 °C.

Fourth, a regeneration solution of 10 mM NaOH was injected twice for 30 s at 30 μl/min flow to remove any non-covalently bound protein after each binding cycle followed by a stabilization period of 40 s to stabilize the baseline.

Signals were detected at a rate of one signal per second. The binding responses (resonance units, RU) of the bivalent, bispecific antibody correlate with the amount of the antigen 1-antigen 2-Fc-fusion and are plotted against the sample concentration range used. The resulting linear plots were analyzed by appropriate computer software (e.g. XLfit4, IDBS Software), which fits a 2-parametric line and hence allows determination of the y-axis intercept as the biological binding activity (=potency) readout. Following a parallel line-transformation, the relative potency of a sample in comparison to the antibody reference standard can be reported (=reportable potency).

The captured antigens 1 and 2 bind to the bivalent, bispecific antibody simultaneously. The targets binding response is used as final assay readout.

The method described above is designed to fulfill the criteria of the USP 1032 for potency release assays, as published by Gassner et al. (Gassner, C., et al. J. Pharm. Biomed. Anal. 102 (2015 ) 144-149). However, measuring a single concentration and plotting it against a calibration curve of a reference standard is sufficient if there is no need to fulfill the USP 1032 criteria.

1. A method for determining the avidity-based binding strength of an at least bivalent, bispecific antibody to its first and second antigen, the method comprising:
determining the avidity-based binding strength of the bivalent, bispecific antibody from the surface-plasmon-resonance (SPR)-signal obtained by applying a solution comprising the bivalent, bispecific antibody to a solid phase to which a covalent fusion polypeptide, which comprises at one terminus the first antigen and at a different, second terminus the second antigen, is conjugated and monitoring the SPR-signal thereafter,
wherein the at least bivalent, bispecific antibody comprises a first binding site specifically binding to a first, non-antibody antigen and a second binding site specifically binding to a second, different, non-antibody antigen.

2. The method according to claim 1 comprising the following steps:
a) capturing a first-antigen-second-antigen-fusion-polypeptide on a solid phase,
b) applying to the solid phase of step a) a first solution comprising the bivalent, bispecific antibody at a first concentration to form a captured first-antigen-second-antigen-fusion-polypeptide-bivalent, bispecific-antibody-complex and determining a first surface-plasmon-resonance-response,
c) dissociating the captured first-antigen-second-antigen-fusion-polypeptide-bivalent, bispecific-antibody-complex and thereby regenerating the solid phase,
d) repeating steps b) and c) at least with a second solution comprising the bivalent, bispecific antibody at a second concentration and determining a second surface-plasmon-resonance-response, whereby all concentrations are different,
e) determining from the surface-plasmon-resonance-responses as determined in the previous steps the avidity-based binding strength of at least the bivalent, bispecific antibody to the first and the second antigen.

3. The method according to claim 1, wherein each covalent fusion polypeptide, which comprises at one terminus the first antigen and at a different, second terminus the second antigen, is conjugated to the solid phase separately.

4. The method according to claim 1, wherein the first antigen is at least a fragment of the first antigen comprising the epitope of the first binding site of the bivalent, bispecific antibody and the second antigen is at least a fragment of the second antigen comprising the epitope of the second binding site of the bivalent, bispecific antibody.

5. The method according to claim 1, wherein the first antigen is different from the second antigen.

6. The method according to claim 1, wherein the solid phase is a surface plasmon resonance chip.

7. The method according to claim 1, wherein the first-antigen-second-antigen-fusion-polypeptide is a heterodimeric polypeptide comprising a first polypeptide, which is a fusion polypeptide of the first antigen and a first antibody heavy chain Fc-region polypeptide comprising a first set of heterodimerizing mutations, and a second polypeptide, which is a fusion polypeptide of the second antigen and a second antibody heavy chain Fc-region polypeptide comprising a second set of heterodimerizing mutations complementary to the first set of heterodimerizing mutations.

8. The method according to claim 7, wherein the first antigen and the second antigen are at the N-terminus of the respective first or second Fc-region polypeptide.

9. The method according to claim 1, wherein the first-antigen-second-antigen-fusion-polypeptide comprise a tag for immobilization to a solid phase.

10. The method according to claim 9, wherein the tag is at the C-terminus of the respective Fc-region polypeptide.

11. The method according to claim 7, wherein the Fc-region is of the human IgG1 isotype.

12. The method according to claim 7, wherein the first and the second set of heterodimerizing mutations are T366W and T366S/L368A/Y407V, respectively, or vice versa.

13. A heterodimeric fusion polypeptide comprising
i) a first polypeptide, and
ii) a second polypeptide,
wherein
the first polypeptide and the second polypeptide are the first and the second antigen of a bispecific antibody which comprises a first binding site that specifically binds to the first polypeptide and a second binding site that specifically binds to the second polypeptide,
the first polypeptide is fused to the N-terminus of a first antibody heavy chain Fc-region polypeptide of the IgG1 subtype,
the second polypeptide is fused to the N-terminus of a second antibody heavy chain Fc-region polypeptide of the IgG1 subtype,
the first and the second heavy chain Fc-region polypeptide form a disulfide-linked heterodimer,
one or both of the heavy chain Fc-region polypeptides comprise a tag for immobilization to a solid phase at its C-terminus,
the first and the second Fc-region polypeptide comprise the mutations T366W and T366S/L368A/Y407V, respectively, and
the first antigen is different from the second antigen.

14. The use of a heterodimeric fusion polypeptide according to claim 13 for the determination of the avidity-based binding strength of a bispecific antibody, which comprises a first binding site specifically binding to a first antigen and a second binding site specifically binding to a second antigen, to said first and second antigen in a surface-plasmon-resonance-method.

15. A method for purifying a bispecific antibody, which comprises a first binding site specifically binding to a first antigen and a second binding site specifically binding to a second antigen, with avidity-based binding to the first and second antigen from product- and/or process-related impurities, comprising the following steps:
a) applying a solution comprising the bispecific antibody with avidity-based binding to the first and second antigen and process- and/or product-related impurities to an affinity chromatography column comprising the heterodimeric fusion polypeptide according to claim 13 as chromatography ligand,
b) optionally washing the column whereby the bispecific antibody with avidity-based binding to the first and second antigen remains bound to the column, and
c) recovering the bispecific antibody with avidity-based binding to the first and second antigen from the column and thereby purifying a bispecific antibody (from product- and/or process-related impurities).

16. A method for assessing the quality of a sample comprising an at least bivalent, bispecific antibody comprising the following steps:
a) separately applying solutions comprising a covalent fusion polypeptide, which comprises at one terminus the first antigen and at a different, second terminus the second antigen of the bivalent, bispecific antibody, at different concentrations to an SPR chip on which the bivalent, bispecific antibody has been immobilized and monitoring the SPR-signal thereafter, and
b) comparing the determined readout with a reference sample and thereby determining the quality of the sample comprising the at least bivalent, bispecific antibody,
wherein the at least bivalent, bispecific antibody comprises a first binding site specifically binding to a first, non-antibody antigen and a second binding site specifically binding to a second, different, non-antibody antigen.

17. The method according to claim 16, wherein the method comprises the following steps:
a) separately applying solutions comprising a covalent fusion polypeptide, which comprises at one terminus the first antigen and at a different, second terminus the second antigen of the bivalent, bispecific antibody, at different concentrations to an SPR chip on which the bivalent, bispecific antibody has been immobilized and monitoring the SPR-signal thereafter,
b) plotting the binding response against the respective sample concentration,
c) fitting the data points of the obtained plot using a 2-parametric line fit and determining the y-axis intercept as readout,
d) comparing the determined readout by parallel-line transformation with that of a reference sample that has been analyzed and processed in the same way,
thereby determining the quality of the sample comprising the at least bivalent, bispecific antibody,
wherein the at least bivalent, bispecific antibody comprises a first binding site specifically binding to a first, non-antibody antigen and a second binding site specifically binding to a second, different, non-antibody antigen.

18. A method for selecting a cell line producing an at least bivalent, bispecific antibody comprising the following steps:
a) providing the individual supernatants of the separate cultivations of the cell lines of a multitude of recombinant mammalian cell lines expressing an at least bivalent, bispecific antibody,
b) for each cell line separately applying solutions comprising a covalent fusion polypeptide, which comprises at one terminus the first antigen and at a different, second terminus the second antigen of the bivalent, bispecific antibody, at different concentrations to an SPR chip on which the bivalent, bispecific antibody from the cultivation supernatant of said cell line has been immobilized and monitoring the SPR-signal thereafter, or vice versa
c) comparing the determined readouts with each other and thereby determining the relative quality of the at least bivalent, bispecific antibody produced by each cell line, and
d) selecting at least one cell line based on the relative quality of the at least bivalent, bispecific antibody produced,
wherein the at least bivalent, bispecific antibody comprises a first binding site specifically binding to a first, non-antibody antigen and a second binding site specifically binding to a second, different, non-antibody antigen.

19. The method according to claim 18, wherein the method comprises the following steps
a) providing the individual supernatants of the separate cultivations of the cell lines of a multitude of recombinant mammalian cell lines expressing an at least bivalent, bispecific antibody,
b) for each cell line separately applying solutions comprising a covalent fusion polypeptide, which comprises at one terminus the first antigen and at a different, second terminus the second antigen of the bivalent, bispecific antibody, at different concentrations to an SPR chip on which the bivalent, bispecific antibody from the cultivation supernatant of said cell line has been immobilized and monitoring the SPR-signal thereafter, or vice versa,
c) plotting the binding response against the respective sample concentration,
d) fitting the data points of the obtained plot using a 2-parametric line fit and determining the y-axis intercept as readout, thereby determining the relative quality of the at least bivalent, bispecific antibody produced by each cell line, and
e) selecting at least one cell line based on the relative quality of the at least bivalent, bispecific antibody produced,
wherein the at least bivalent, bispecific antibody comprises a first binding site specifically binding to a first, non-antibody antigen and a second binding site specifically binding to a second, different, non-antibody antigen.
